(19) United States
(12) Patent Application Publication
(10) Pub. No.: US 2026/0308084 A1
(43) Pub. Date: Oct. 8, 2026

(54) Undirected, Z-selective C-H Functionalization of Unactivated Alkenes

(71) Applicant: Wisconsin Alumni Research Foundation, Madison, WI (US)

(72) Inventors: Zachary Wickens, Madison, WI (US); Peter Verardi, Madison, WI (US); Karina Targos, Carmel, IN (US); Elizabeth Ryutov, Madison, WI (US)

(73) Assignee: Wisconsin Alumni Research Foundation, Madison, WI (US)

(21) Appl. No.: 19/091,428

(22) Filed: Mar. 26, 2025

(51) Int. Cl. C07C 2/80; C07D 339/08

(52) U.S. Cl. CPC C07C 2/80; C07D 339/08; C07C 2523/44

(57) ABSTRACT

A method to make Z-alkene-containing compounds by electrochemically reacting a 1-alkene with a thianthrene-containing compound to yield a first intermediate and subjecting the first intermediate to an E2 elimination reaction to yield a product comprising a Z-alkenyl moiety in diastereomeric excess.

### FEDERAL FUNDING STATEMENT

This invention was made with government support under GM149674 awarded by the National Institutes of Health. The government has certain rights in the invention.

### BACKGROUND

Selective transformation of C—H bonds is a longstanding goal of organic synthesis. Decades of research have delivered an array of mechanistically diverse strategies to directly replace these classically inert C—H bonds (bond-dissociation energy=95-110 kcal/mol; pKa=45-50) with new C—C bonds. See, for example, Davies, Du Bois, Yu, Quan. “C—H Functionalization in Organic Synthesis,” Chem. Soc. Rev. 2011, 40, 1855-1856 and Hartwig, “Catalyst-Controlled Site-Selective Bond Activation,” Acc. Chem. Res. 2017, 50, 549-555. Within this domain, alkenyl C—H functionalization has experienced substantial development (Beletskaya, Cheprakov, “The Heck Reaction as a Sharpening Stone of Palladium Catalysis,” Chem. Rev. 2000, 100, 3009-3066. Tang, Liu, Liu, Lei, “Olefinic C—H Functionalization through Radical Alkenylation,” Chem. Soc. Rev. 2015, 44, 1070-1082.) However, highly Z-selective reactions remain elusive. For example, a Z-selective variant of the Heck reaction has yet to arise despite over half a century of innovation on this Nobel Prize-winning reaction. (See, for example, Hirabayashi, Ando, Kawashima, Nishihara, Mori, & Hiyama, “Novel Carbon-Carbon Bond Formation through Mizoroki-Heck Type Reaction of Silanols and Organotin Compounds,” Bull. Chem. Soc. Jpn. 2000, 73, 1409-1417.

Indeed, Z-selective C—H olefination of terminal alkenes presents a formidable challenge due to the inextricable introduction of unfavorable allylic-1,3 strain upon functionalization. See FIG. 1, Panel A. See also Hoffman, “Allylic 1,3-Strain as a Controlling Factor in Stereoselective Transformations,” Chem. Rev. 1989, 89, 1841-1860 and Lu, Goh, Maraswami, Jia, Tian, & Loh, “Recent Advances in Alkenyl Sp2 C—H and C—F Bond Functionalizations: Scope, Mechanism, and Applications,” Chem. Rev. 2022, 122, 17479-17646. Any Z-selective method must somehow offset this energetic penalty. Consequently, while a handful of Z-selective alkenyl C—H olefination reactions have emerged, they categorically rely on direct substitution of the alkene with strongly coordinating directing groups to dictate stereoselectivity. See Lu et al., supra, and Zhang, Lu, Shen, Xu, Ding, & Zhong, “Recent Advances in Chelation-Assisted Site- and Stereoselective Alkenyl C—H Functionalization,” Chem. Soc. Rev. 2021, 50, 3263-3314. This strategy intrinsically limits the viable substrates to a small pool of rigid, activated alkenes bearing specific auxiliaries. Again, see Lu et al., supra. Therefore, there exists a long-felt and unmet need for a fundamentally new method to promote the undirected Z-selective C—H functionalization of terminal alkenes.

### SUMMARY OF THE INVENTION

Disclosed herein is a method to Z-alkenes via a novel, undirected, Z-selective C—H functionalization reaction using a thianthrenium adduct.

Alkenyl C—H functionalization represents a premier tactic to access complex olefinic products from inexpensive feedstocks. While E-selective alkenyl C—H functionalization (e.g., Heck) is well-established, access to the thermodynamically disfavored Z-isomer via this strategy remains elusive. Disclosed herein is an undirected, electrochemical method to Z-selective C—H functionalization of unactivated terminal alkenes. The method enables concise preparation of Z-alkene products. Key to the strategy is the discovery that di-cationic bis-thianthrenium adducts undergo a highly stereoselective E2 reaction to form Z-alkenyl thianthrenium salts. Paired electrolysis in an undivided cell promotes bis-adduct formation to deliver the resultant Z-alkenyl thianthrenium salts in high yield. These stereo-defined electrophiles serve as synthetic equivalents to diverse C—C linkages via catalytic cross-coupling reactions. Computational investigations of bis-adduct elimination reveal that p-p interactions between the two thianthrenium units selectively stabilize the pro-Z transition structure.

### Abbreviations and Definitions

All references to singular characteristics or limitations of the disclosed ball valve shall include the corresponding plural characteristic or limitation, and vice-versa, unless otherwise specified or clearly implied to the contrary by the context in which the reference is made. The indefinite articles “a” and “an” mean “one or more.”

All combinations of method steps disclosed herein can be performed in any order, unless otherwise specified or clearly implied to the contrary by the context in which the referenced combination is made.

The method disclosed herein can comprise, consist of, or consist essentially of the steps and elements described herein, as well as any additional or optional steps, elements, components, or limitations described herein or otherwise useful in liquid handling equipment.

The word “or” is used inclusively and means “and/or.”

Disastereomeric excess (d.e.) is defined as the absolute difference between the mole fraction of each diastereomer (E vs. Z), expressed as a fraction:

$$\% d . e . = ( \vert {F}_{Z} - {F}_{E} \vert ) \times 100 ( where {F}_{Z} + {F}_{E} = 1 ) .$$

HER=hydrogen evolution reaction.

The phrase “thianthrene-containing compound” is defined broadly herein to include any compound of any description that incorporates a thianthrene moiety. The definition explicitly includes, but is not limited to, compounds of the following structure:

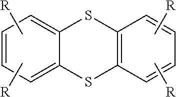

wherein each R is independently selected from the group consisting of hydrogen, halogen, hydroxy, amino, and substituted or unsubstituted, linear or branched C1-C6 alkyl. Substituted alkyl groups may include one or more substituents selected from the group consisting of halogen, hydroxy, and amino.

The phrase “weak base” is defined herein as any base having a pKb greater than about 9. Weak bases include but are not limited to salts of conjugated bases of weak acids. These may include, for example, the salts of conjugated bases of carboxylic acids and/or phosphoric acids, e.g., carbonates, bicarbonates, acetates, citrates, halogenated versions of the foregoing, and the like. Specific, non-limiting examples of weak bases include salts of trifluoroacetate (preferred), ammonium hydroxide, sodium carbonate, potassium carbonate, calcium carbonate, magnesium hydroxide, ferric hydroxide, as well as monoethanolamine, diethanolamine, triethanolamine, ethylenediamine, cysteine, N-methylethanolamine, N-methyldiethanolamine, dimethylethanolamine, N,N-diisopropylaminoethanol, methyl diethanolamine, bis-tris methane, meglumine, aminoethylethanolamine, N-methylaminoethanol, aminoethoxyethanol, dimethylaminoethoxyethanol, isopropanolamine, diisopropanolamine, aminopropyldiethanolamine, N,N-dimethylpropanolamine, N-methylpropanolamine, 1-amino-2-propanol, 2-amino-1-butanol, isobutanolamine, and the like.

### BRIEF DESCRIPTION OF DRAWINGS

FIG. 1 is a set of reaction schemes illustrating the challenges in Z-olefin synthesis. Panel A depicts a simplified rendition of prior art directly C—H functionalization to yield Z-olefins. Panel B depicts the novel method disclosed herein, namely a Z-selective E2 reaction followed by a metal-catalyzed cross-coupling reaction to yield Z-olefins. Specifically shown in Panel B is an undirected C—H functionalization of unactivated alkenes using thianthrenium salts.

FIG. 2 is a set of reaction schemes showing the underpinnings of the present method. Panel A depicts the novel highly Z-selective bis-adduct elimination disclosed herein. Panel B depicts paired electrolysis in an undivided cell to drive Z-selective alkene thianthrenation.

FIG. 3 depicts, in Panel A, various Z-alkenyl thianthrenium electrophiles and, in Panel B, an exemplary, decagram-scale Z-selective C—H functionalization reaction according to the present method.

FIG. 4 depicts, in Panel A, exemplary cross-coupling reactions according to the method disclosed herein that pass through the Z-alkenyl thianthrenium intermediate. Panel B depicts exemplary reactions using the present method to make bioactive natural products via Z-selective C—H functionalization.

FIG. 5, Panel A, is a proposed mechanism to account for the Z-selectivity of the disclosed method. (The method, however, is not limited to any specific mechanism.) Panel B depicts proposed stabilizing interactions that contribute to the observed Z-selectivity.

FIG. 6 is a histogram showing yield versus solvent for the reaction disclosed herein

FIG. 7 is a histogram showing yield versus electrolyte for the reaction disclosed herein

FIG. 8 is a histogram showing yield versus reactant concentration for the reaction disclosed herein

### DETAILED DESCRIPTION OF THE INVENTION

Newly developed and disclosed herein is a method of making Z-alkene-containing compounds. The method is undirected, electrochemical, and predictably yields alkene-containing compounds wherein the alkene bond formed by the reaction is in the Z conformation. At the heart of the method is the formation of a di-cationic, bis-thianthrenium adduct that undergoes a highly stereo-selective E2 elimination reaction to yield a Z-alkenyl thianthrenium salt. The salts can then be reacted to yield a wide range of Z-alkenyl-containing products.

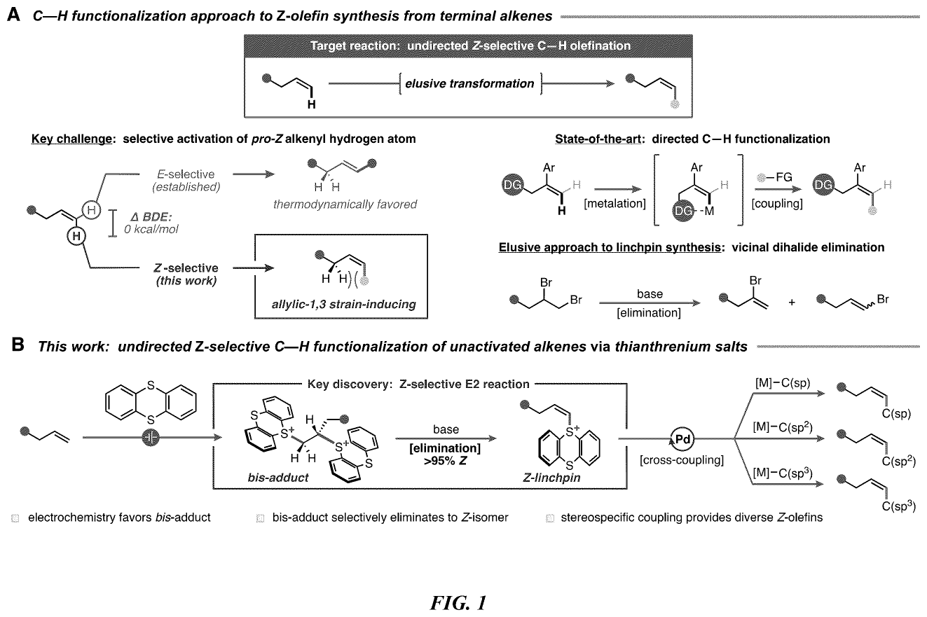

Disclosed herein is a novel method for Z-selective C—H functionalization enabled by installing a diversifiable handle at the pro-Z position of terminal alkenes. The resulting intermediate may then be driven to a desired product by, for example, downstream cross-coupling reactions. Despite its synthetic appeal, such a linchpin approach to Z-selective C—H functionalization has (up to now) proven elusive. To date, all existing reports rely on coordinating auxiliaries to achieve Z-selectivity. Emerging metathesis-based approaches to Z-alkenyl halide synthesis bypass C—H cleavage entirely, leaving this key challenge unaddressed. In principle, Z-selective elimination of a vicinal dihalide derived from a terminal alkene could provide the desired Z-linchpin. However, this seemingly elementary reaction does not exist; it does not happen. While dihalides can be prepared from terminal alkenes, they eliminate unselectively to mixtures of regio- and stereoisomers. Overall, a practical elimination reaction that delivers Z-alkenyl linchpins from C(sp²)-H bonds would not only be immediately synthetically useful but would also address a fundamental challenge in organic synthesis. Disclosed herein is a novel method for Z-selective C—H functionalization, which exploits a newfound mechanistic discovery: the first Z-selective E2 reaction that does not rely on the transfer of starting material stereochemistry. See FIG. 1, Panel B, which presents as schematic diagram of the new approach. The elimination of bis-alkene-thianthrenium adducts was found to yield Z-alkenyl thianthrenium salts (called the “Z-linchpin” in FIG. 1, Panel B). The Z-linchpin then serves as a stable, stereo-defined alkenyl intermediate for diverse downstream coupling reactions (such as the exemplary, palladium-catalyzed cross-coupling reactions illustrated in FIG. 1, Panel B).

Briefly summarizing FIG. 1, Panel B, a starting 1-alkene (i.e., an alpha olefin) is reacted electrochemically with a thianthrene-containing reagent to yield an intermediate. While not being limited to any specific mechanism or underlying phenomenon, it is thought that the reaction yields the di-cationic bis adduct shown at the left within the boxed portion of FIG. 1, Panel B. Treating the bis adduct with base results in a stereoselective E2 elimination of one of the thianthrene moieties to yield the “Z-linchpin,” a thianthrenium salt bearing an appended Z-alkenyl moiety. Surprisingly, the Z isomer is formed in greater than 95% excess. The Z-linchpin can then be reacted in any number of ways to yield desired compounds that incorporate the Z-alkenyl moiety produced by the reaction. See the far-right of FIG. 1, Panel B.

### Discovery of a Z-Selective Elimination:

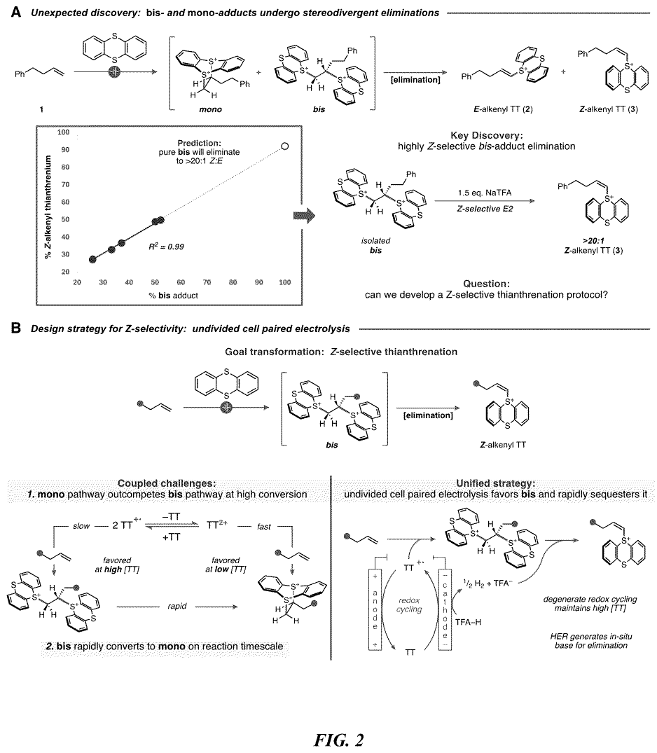

While studying the elimination behavior of electrogenerated mono- and bis-thianthrenium adducts, an unambiguous trend was observed between the ratio of these two intermediatespecies (bis:mono) and the diastereomeric ratio of the resulting alkenyl thianthrenium products. (See FIG. 1, Panel A; and FIG. 2, Panel A).

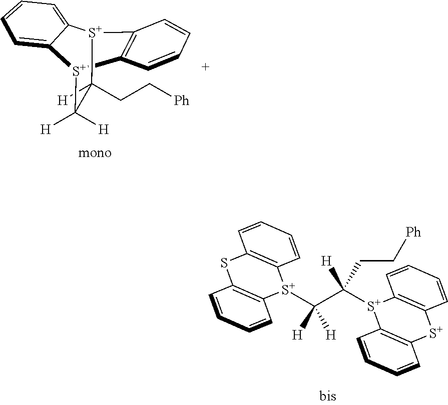

While these conditions were not selective to the bis-adduct, it was found that bis-enriched adduct mixtures resulted in higher percentages of Z-product following elimination. In short, the present inventors found that the bis-adduct preferentially eliminates to the Z-alkenyl thianthrenium product. To directly evaluate the bis-adduct elimination stereoselectivity, the small amount of elimination product formed under electrolysis was isolated by iterative recrystallization. Upon treatment of the pure bis-adduct with a weak base (trifluoroacetate), rapid and exquisitely stereoselective elimination to Z-alkenyl thianthrenium salt 3 (>20:1 Z:E) was observed. This stereoselective process is necessarily and fundamentally distinct from stereospecific E2 reactions, in which the stereochemistry of the starting material dictates the final alkene diastereomer. See, for example, Kuang, Senboku, & Tokuda, “Convenient and Stereoselective Synthesis of (Z)-1-Bromo-1-Alkenes by Microwave-Induced Reaction,” Tet. Lett. 2001, 42, 3893-3896. Here, a terminal alkene starting material (which is not stereoisomeric) passes through the bis-adduct to yield the “Z-linchpin” in diastereomeric excess. The method specifically encompasses running the reaction to yield the “Z-linchpin” in a diastereomeric excess of at least 50%, at least 60%, at least 70%, and least 80%, at least 90%, or at least 95%.

To the inventors' knowledge, the bis elimination method disclosed herein is the first report of a Z-selective E2 reaction. While bis-thianthrenium adducts are not formed under S-oxide-based chemical thianthrenation, the present inventors also determined that a bis-selective electrolysis delivered the Z-selective thianthrenation. This, the bis-selective electrochemical reaction disclosed herein yields a stereo-defined linchpin for Z-alkene synthesis via stereospecific cross-coupling.

Given that bis and mono arise from distinct mechanistic pathways, it was hypothesized that the electrochemical parameters could be tuned to deliver high concentration of the bis intermediate. The initial efforts were stymied by two key challenges: (1) the mono-forming pathway occurred at a faster rate than the bis-forming pathway, and (2) bis was rapidly converted to mono on the timescale of adduct generation. To address these problems, it was envisioned to suppress mono formation and drive the elimination reaction of the bis isomer in-situ to prevent its conversion to mono. It was hypothesized that at high [π](i.e., E diastereomer=compound 2, Z diastereomer=compound 3; see FIG. 2, Panel A, top-right; structures of 2 and 3 copied below), favorable comproportionation of π and π²⁺ could suppress formation of the mono intermediate.

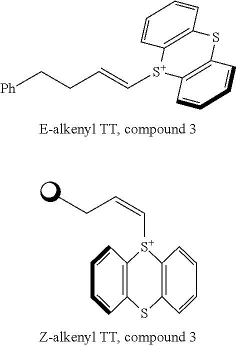

Due to the incompatibility of oxidized π with many bases, high base loading could disrupt both pathways. While careful reaction engineering (e.g., increased π loadings and slow addition of base) could potentially control each of the necessary concentrations, paired electrolysis in an undivided cell afforded an elegant solution to both challenges. Specifically, by housing the anode and cathode in the same compartment, anodically-generated π^{•+} underwent off-pathway reduction at the cathode to maintain a high steady-state [π]. As a result of this degenerate redox cycling, any π²⁺ generated under the reaction conditions rapidly comproportionates with π to restore π^{•+}. Furthermore, a hydrogen evolution reaction (HER) generates trifluoroacetate at the same rate as π is oxidized. This in-situ base eliminated bis to Z-alkenyl thianthrenium faster than its conversion to the mono intermediate. See FIG. 2, Panel B, bottom. Gratifyingly, switching from a divided cell to an undivided cell and employing a Ni foam cathode to improve HER efficiency afforded the desired Z-alkenyl thianthrenium in modest diastereoselectivity. Optimization of solvent, current, and charge under undivided cell electrolysis delivered final conditions that furnished the Z-alkenyl thianthrenium salt 3 (see FIG. 2) in both high yield and diastereoselectivity.

### Scope and Synthetic Utility of Z-Alkenyl Thianthrenium Salts:

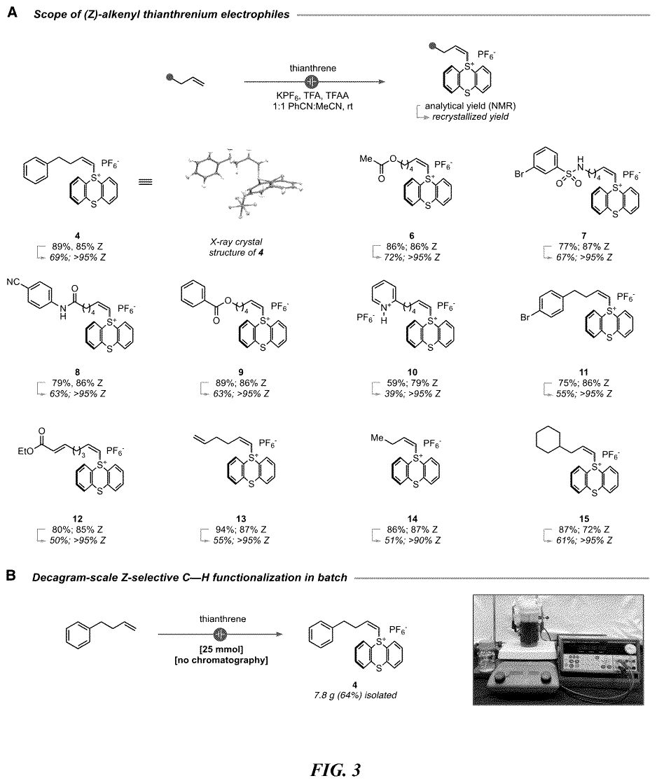

The scope of alkenes amenable to this Z-selective thianthrenation protocol was then explored. It was found that a wide range of terminal alkenes are readily transformed into Z-alkenyl thianthrenium salts bearing diverse functional groups, including esters, sulfonamides, amides, nitriles, pyridines, and aryl bromides (6-11). See FIG. 3. Notably, unconjugated dienes 12 and 13 undergo selective monofunctionalization to provide useful building blocks with two orthogonal sites for further derivatization. Excitingly, the undivided electrolysis conditions are easily adapted to engage gaseous feedstocks such as 1-butene to provide the corresponding alkenyl thianthrenium salts (14) with high diastereoselectivity. Additionally, alkenes with proximal steric bulk underwent Z-selective thianthrenation, albeit with slightly diminished Z-selectivity (15). It was found that each of these Z-alkenyl thianthrenium salts was crystalline and a simple recrystallization procedure furnished diastereopure material. Underscoring its practical synthetic utility, this undivided cell thianthrenation was readily scaled in batch to deliver multigram quantities of pure Z-alkenyl thianthrenium 4 following recrystallization.

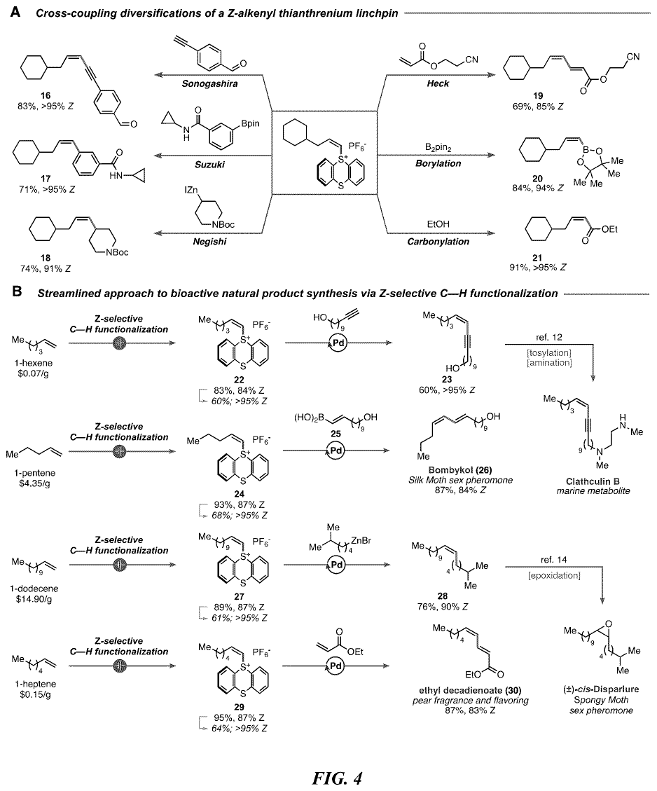

The Z-selective C—H thianthrenation method disclosed herein provides an electrophilic linchpin for rapidly synthesizing a diverse array of Z-alkenyl products via stereospecific cross-coupling. However, the prior literature indicates that alkenyl thianthrenium salts undergo isomerization under basic conditions. See Targos, Gogoi, Renteria-Gomez, Kim, Gutierrez, and Wickens, “Mechanism of Z-Selective Allylic Functionalization via Thianthrenium Salts,” ChemRxiv Jan. 17, 2024. Wang, Targos, and Wickens, “Electrochemical Synthesis of Allylic Amines from Alkenes and Amines,” J. Am. Chem. Soc. 2021, 143, 21503-21510. The stereospecificity of thianthrenium cross-couplings was thus unestablished and unknown. It was unexpectedly, unpredictably, and very pleasingly discovered that the stereochemistry of model alkenyl thianthrenium 15 was readily translated to a variety of C—C bonds with minimal deviations from standard cross-coupling conditions. See FIG. 4, Panel A. Here, working examples of Pd-catalyzed Sonogashira, Suzuki, and Negishi coupling protocols each afforded the corresponding C(sp), C(sp²), and C(sp³) cross-coupled products 16-18 with excellent retention of the Z alkene geometry. A Heck reaction delivered the corresponding Z,E-diene product 19 with minimal erosion of diastereoselectivity despite forming an activated diene product and proceeding through a Pd—H intermediate known to promote Z- to E-alkene isomerization. Borylation transposed the polarity of the Z-alkenyl thianthrenium handle to furnish the nucleophilic Z-alkenyl building block 20. (Xie, Zhu, & Huang, “Cu-Catalyzed Highly Selective Silylation and Borylation of Alkenylsulfonium Salts,” Org. Chem. Front. 2021, 8 (20), 5699-5704.) Finally a Pd-catalyzed carbonylation furnished Z-acrylate product 21. This new reaction for alkenyl thianthrenium salts demonstrates that these intermediates can serve as Z-alkenyl pseudohalides for a wide range of other classic coupling reactions.

The Z-selective C—H functionalization method disclosed and claimed herein enables making a wide array of synthetic targets easily and without the need for diastereomeric starting materials. By way of a working example, see FIG. 4, Panel B. The Z-enyne core of Clathculin B, a marine natural product, presents an intuitive Sonogashira disconnection. Using the new C—H method disclosed herein, 1-hexene ($0.07/g) underwent Z-selective thianthrenation (22) and cross-coupling with commercially available 10-undecyn-1-ol to provide key intermediate 23 on the path to Clathculin B. (Hoye, Anderson, Brown, & Schultz, “Total Synthesis of Clathculins A and B,” J. Org. Chem. 2010, 75, 7400-7403.) Similarly, Z-selective thianthrenation of 1-pentene ($4.35/g) provided Z-linchpin 24, setting the stage for a Suzuki coupling with alkenylboronic acid 25 to furnish the Silk Moth sex pheromone Bombykol (26) in excellent yield in three overall steps. (Miyaura & Suginome, “New Stereospecific Syntheses of Pheromone Bombykol and Its Three Geometrical Isomers,” Tetrahedron 1983, 39, 3271-3277.) A C(sp²)-C(sp³) Negishi coupling of Z-dodecenyl thianthrenium salt 27 and isoheptylzinc bromide was successfully used to provide 28 as a key step in the synthesis of Spongy Moth sex pheromone (±)-cis-Disparlure. (Herbert, Marx, Pederson, & Grubbs, “Concise Syntheses of Insect Pheromones Using Z-Selective Cross Metathesis,” Angew. Chem. Int. Ed. 2013, 52, 310-314.) Finally, ethyl decadienoate (30), an important fragrance, was synthesized in only two steps via a Heck reaction of ethyl acrylate and Z-heptenyl thianthrenium salt 29. (Tsuboi, Masuda, Mimura, & Takeda, “Ethyl (E/Z)-2,4-Decadienoate,” Org. Synth. 1988, 66.) Given the success demonstrated here and the scalability of the disclosed Z-selective thianthrenation method, the method can be used to prepare a wide range of Z-alkene targets.

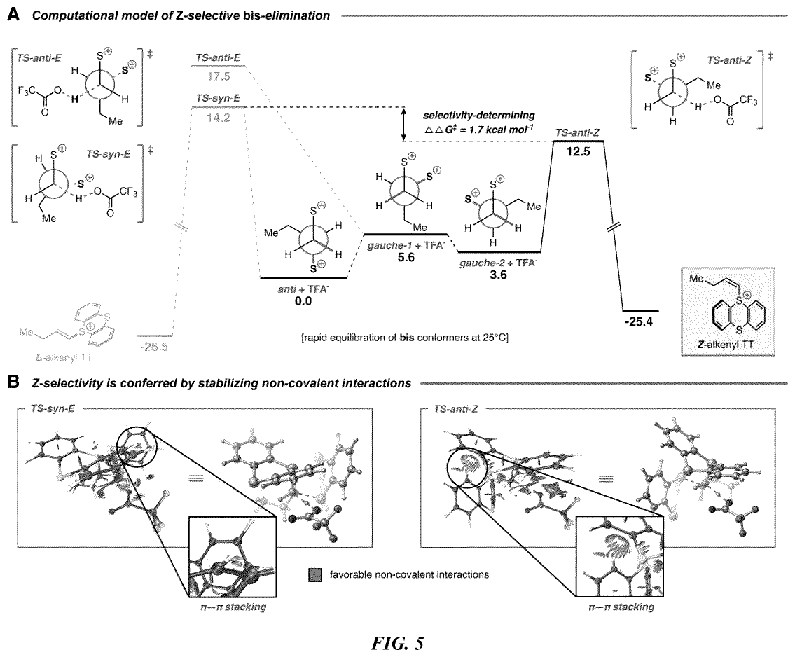

Having demonstrated the synthetic value of this new Z-selective C—H functionalization method, the mechanistic origins of the high elimination stereoselectivity was investigated. Note that the method, however, is not limited in any way to any specific mechanism or underlying phenomena. See FIG. 5. Using 1-butene as a representative substrate, the three ground-state bis conformations were modeled as encounter complexes with trifluoroacetate. Rapid equilibration of these conformers at room temperature implicates a Curtin-Hammett scenario wherein diastereoselectivity is controlled by differences in elimination transition structure energies. Of the two elimination pathways displaying the correct orbital alignment for a classic E2-type anti-elimination, TS-anti-Z is lower in energy than TS-anti-E. To probe the origin of the Z-selectivity, a non-covalent interaction (NCI) analysis of each transition structure was performed. These revealed stabilizing p-p interactions between the two aromatic rings of the thianthrenium units in TS-anti-Z. No pro-E anti-periplanar transition structures that restored these interactions converged. Without being restricted to any given mechanism or underlying phenomenon, presumably this is due to steric clashes between the thianthrenium unit and the alkyl substituent in TS-anti-E. In fact, a sterically congested syn-elimination (TS-syn-E) is the only way to reengage in these stabilizing interactions. While this pathway remains higher in energy than TS-anti-Z, TS-syn-E is the lowest energy pathway to an E-product. Taken together, these results indicate that TS-anti-Z, very counterintuitively, is the least sterically hindered transition structure that maintains important stabilizing p-p contacts. This analysis demonstrates that stabilizing non-covalent interactions between the thianthrenium moieties are sufficiently strong to offset the steric penalties endemic to Z-selective elimination transition structures.

In summary, disclosed herein is an undirected, Z-selective C—H functionalization of unactivated alkenes. Key to the method is the discovery that bis-thianthrenium adducts eliminate highly stereoselectively to the corresponding Z-alkenyl thianthrenium salts. Mechanistic studies indicate that a key p-p interaction stabilizes the Z-selective elimination transition structure that affords the observed Z-alkenyl thianthrenium product. Using an undivided electrochemical cell affords a reaction pathway leading to the desired bis-adduct, which then causes Z-selective thianthrenation. These Z-alkenyl thianthrenium salts are useful in stereospecific cross-couplings to access a variety of valuable Z-alkenyl building blocks and natural products.

### Examples

### General Methods and Materials:

DMF, THF, Et20, and DCM were dried by passing through activated alumina columns. Potassium hexafluorophosphate (KPF₆) was dried under vacuum prior to use. Unless otherwise noted, other commercially available reagents were used as received. Crude mixtures were evaluated by thinlayer chromatography using EMD/Merck silica gel 60 F254 pre-coated plates (0.25 mm) and were visualized by UV and/or KMnO₄ and Ninhydrin staining. Flash chromatography was performed with a Biotage Isolera One automated chromatography system with re-packed silica columns (technical grade silica, pore size 60 Å, 230-400 mesh particle size, 40-63 particle size) unless otherwise noted. Purified materials were dried in vacuo (0.050 Torr) to remove trace solvent. ¹H, ¹³C, ¹⁹F, and ³¹P spectra were taken using a Bruker Avance400 with a BBFO Probe, a Bruker Avance-500 with a DCH Cryoprobe, or a Bruker Avance Neo 500 with a Prodigy probe. NMR data are reported relative to residual CHCl₃ (¹H, δ=7.26 ppm), CDCl₃ (¹³C, δ=77.16 ppm), CH₃CN (¹H, δ=1.94 ppm), CD₃CN (¹³C, δ=118.26 ppm), CH₃OH (¹H, δ=3.31 ppm), or CD₃OD (¹³C, δ=49.00 ppm). Data for ¹H-NMR spectra are reported as follows: chemical shift (6 ppm) (multiplicity, coupling constant (Hz), integration). Multiplicity and qualifier abbreviations are as follows: s=singlet, d=doublet, t=triplet, q=quartet, p=pentet, hept=heptet, m=multiplet, br=broad. All NMR yields were determined via reference against an internal standard (dibromomethane or mesitylene for ¹H-NMR). Mass spectrometry data was collected on a Thermo Scientific Q Exactive Plus Mass Spectrometer, a Waters Acquity™ LCMS, or a Shimadzu GCMSTQ8040NX®.

Abbreviations: Boc—tert-butyl carbamate, DCM—dichloromethane, EtOAc—ethyl acetate, MeCN—acetonitrile, MeOH—methanol, RVC—reticulated vitreous carbon, SS—stainless steel, Ph—phenyl, TFA—trifluoroacetic acid, THF—tetrahydrofuran, RSM—returned starting material, PC—propylene carbonate, DIPEA—diisopropylethylamine.

### Electrochemical Methods and Materials:

3.1 mmol scale constant current electrolysis experiments were carried out using a Jesverty 30 Volt DC Power Supply (purchasable on Amazon, ASIN: B09YSJQWRG). 25 mmol scale constant current electrolysis experiments were carried out using a Keithley 2230-30-1 Triple Channel DC Power Supply. 0.25 mmol (undivided cell electrolysis optimization) and 0.4 mmol scale (divided cell electrolysis optimization and mechanistic studies) constant current electrolysis experiments were carried out using a low current power supply which was externally calibrated with a multimeter using a 10-Ohm resistor.

### Exemplary Procedures/Working Examples

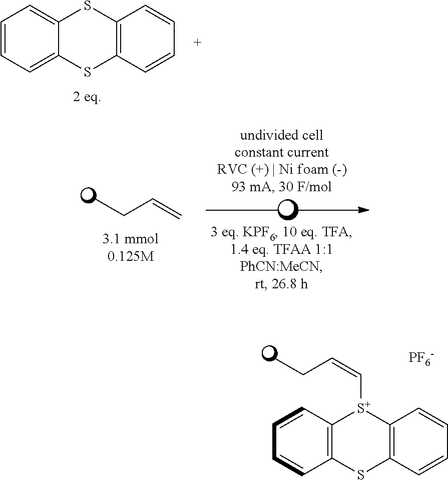

General Procedure A: 3.1 mmol scale batch electrolysis. To an oven-dried, 50-mL, three-necked round-bottom flask equipped with a stir bar were added thianthrene (1.34 g, 6.2 mmol, 2.0 eq.) and KPF₆ (1.71 g, 9.3 mmol, 3.0 eq.). The flask was fitted on either side with rubber septa containing a pencil/RVC anode assembly (electrode dimensions: 1.5×1×1 cm) and a stainless steel wire/Ni foam cathode assembly (electrode dimensions: 2×2 cm, folded in half) and a rubber septum in the center neck. The flask was flushed with N₂, then charged sequentially with anhydrous MeCN (12.5 mL), anhydrous PhCN (12.5 mL), alkene (3.1 mmol), trifluoroacetic acid (2.37 mL, 31 mmol, 10.0 eq.), and trifluoroacetic anhydride (603 μL, 4.3 mmol, 1.4 eq.). Banana leads from the power supply were connected to the electrodes, and the contents of the flask were electrolyzed at a constant current of 93 mA (J=14.8 mA/cm²) for 26.8 h (30 F/mol alkene) with vigorous stirring at room temperature, during which time an intense purple color developed. At completion of electrolysis, the leads were disconnected, and the contents of the flask were concentrated under reduced pressure. The residue was dissolved in CH₂Cl₂ (40 mL) and washed with 5% aq. KPF₆, extracting an additional two times with DCM (20 mL). The combined organic extracts were washed with 5% aq. KPF₆, dried over Na₂SO₄, and concentrated under reduced pressure. To the residue was added 50 mL of diethyl ether, and the mixture was stored overnight in a freezer. The resulting precipitate was filtered in vacuo and then purified by either flash column chromatography to afford a Z-selective mixture of alkenyl thianthrenium isomers or recrystallization to afford diastereopure (>20:1 Z:E) Z-alkenyl thianthrenium salt. Note: Taking no precautions to exclude air and moisture (no N₂ flush, non-dried glassware and solvents) resulted in no change to the reaction yield and selectivity for the model substrate 4-phenylbutene (90%, 85% Z).

Modification of 3.1 mmol scale protocol to accommodate gaseous alkenes: The reaction was conducted using thianthrene as the limiting reagent at a 6.2 mmol scale. After charging the flask with MeCN and PhCN, the resulting solution was sparged for 5 min with a balloon of gaseous alkene, and a full balloon was kept in the headspace of the electrochemical cell during electrolysis (8 F/mol instead of 30 F/mol). A color change from purple to hot pink indicates full consumption of thianthrene and signals the end of electrolysis.

25 mmol scale batch electrolysis: To an oven-dried, tall-form 300-mL beaker equipped with a magnetic stir bar were added thianthrene (10.72 g, 49.6 mmol, 2 eq.) and KPF₆ (13.68 g, 74.4 mmol, 3.0 eq.). The beaker was then charged sequentially with MeCN (100 mL), benzonitrile (100 mL), 4-phenyl-1-butene (3.73 mL, 24.8 mmol), trifluoroacetic acid (19.0 mL, 248 mmol, 10.0 eq), and trifluoroacetic anhydride (7.29 g, 34.4 mmol, 1.4 eq.). The beaker was fitted with an electrode assembly consisting of a machined PTFE cap designed by Kaid Harper (Baran, P. et al. J. Am. Chem. Soc. 2022, 144, 5762-5768) with graphite anodes (electrode dimensions: 0.6×8.8 cm) and stainless-steel cathodes (electrode dimensions: 0.6×8.8 cm), with a total combined wetted area of 107.23 cm². The contents of the beaker were sparged with MeCN-saturated N₂ for 30 min prior to and during electrolysis. The contents of the beaker were electrolyzed at a constant current of 400 mA (J=7.46 mA/cm²) for 30 h (18.05 F/mol alkene) with vigorous stirring at room temperature, during which time an intense purple color developed. At completion of electrolysis, the reaction mixture was worked up and the crude product purified according to General Procedure A.

### Undivided Cell Electrolysis Optimization:

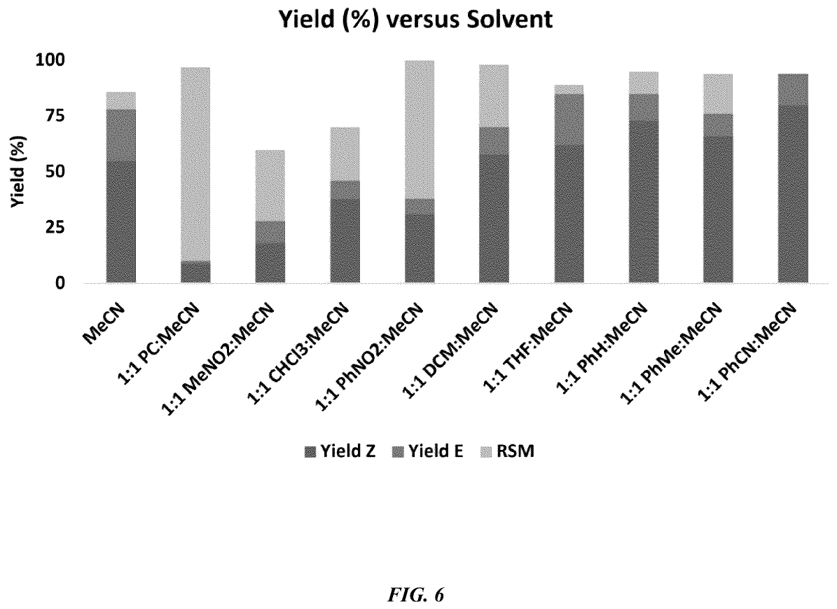

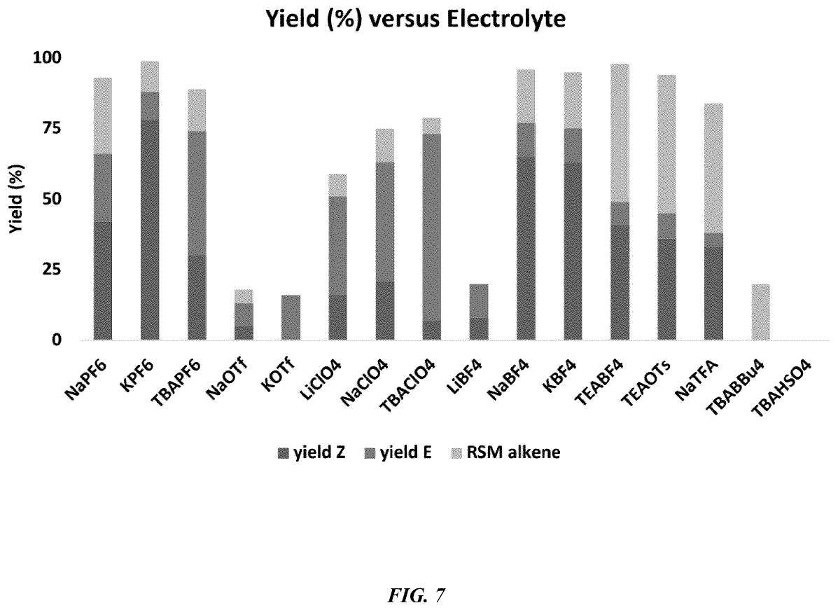

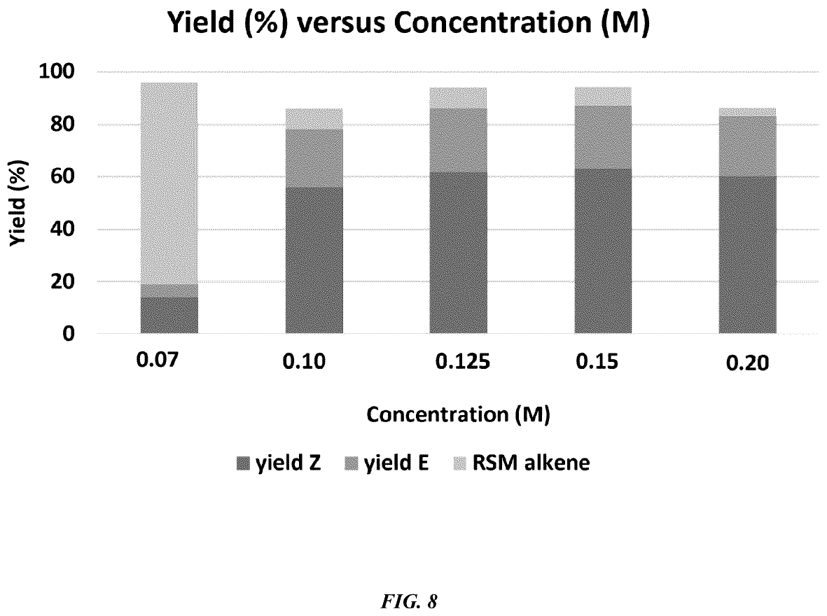

The electrolysis procedures described immediately above were then tested using a host of different solvents, electrolytes, and concentrations. Product yield (%) versus different solvents used is presented in FIG. 6. Product yield (%) versus different elecrolytes used is presented in FIG. 7. Yield (%) versus reactant concentration is shown in FIG. 8.

The electrochemical protocol disclosed herein tolerated a wide variety of functional groups. Crude yields of six additional alkenyl thianthrenium entries are given in Table 1. The results are crude NMR yields. The reactions were conducted on a 3.1 M scale.

TABLE 1

| | Crude NMR | % |
|---|---|---|
| Alkenyl Thianthronium Product | Yield (%), Z:E | RSM |
| 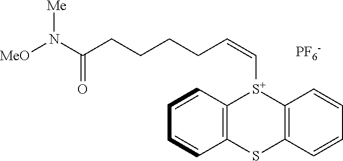 | 71, 87:13 Z:E | 0 |
| 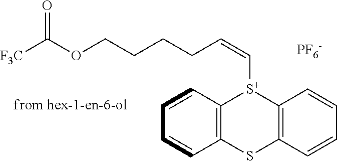 | 71, 80:20 Z:E | 30 |
| 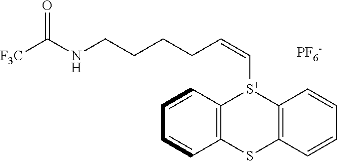 | 87, 87:13 Z:E | 0 |
| 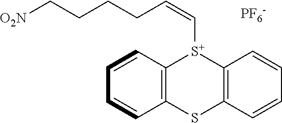 | 82, 87:13 Z:E | 5 |
| 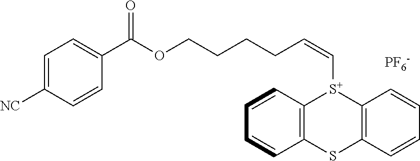 | 90, 85:15 Z:E | 4 |
| 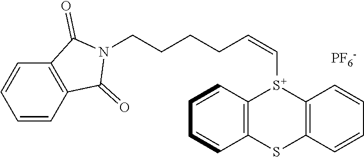 | 77, 87:13 ZE | 0 |

Dicationic adduct elimination studies: to an oven-dried, divided electrochemical cell equipped with rice grain stir bars were added KPF₆ (295 mg, 1.60 mmol, 4.0 eq.) to both compartments and thianthrene (varying amounts) to the anode compartment. The cell was equipped with two septa containing a pencil/RVC anode assembly (electrode dimensions: 0.5×0.5×0.5 cm) and a stainless-steel wire/Ni foam cathode assembly (electrode dimensions: 1×1 cm, folded in half) connected by Teflon tubing to equalize pressure. The cell was flushed with N₂, then each compartment was charged with anhydrous solvent (varying solvent identity) The anode compartment was charged with 4-phenylbutene (60 μL, 0.40 mmol) and trifluoroacetic acid (61 μL, 0.8 mmol, 2.0 eq.), and the cathode compartment was charged with trifluoroacetic acid (153 μL, 2.0 mmol, 5.0 eq.). Banana leads from the power supply were connected to the electrodes, and the contents of the flask were subjected to constant current electrolysis (varying rate and charge, see Table 2 below) with vigorous stirring at room temperature, during which time an intense purple color developed in the anode compartment. At completion of electrolysis, the leads were disconnected, and the anode compartment was charged with dibromomethane standard (28 μL, 0.40 mmol, 1.0 eq.). After stirring for 1 min, a 100-ptL aliquot of the anolyte was removed and diluted with CD₃CN in an NMR tube for quantitative ¹H-NMR analysis (10-second relaxation delay between pulses). The same NMR tube was then charged with sodium trifluoroacetate (10 mg) and shaken vigorously to quantitatively eliminate the dicationic adducts, and a second quantitative ¹H-NMR spectrum was collected (10-second relaxation delay between pulses).

TABLE 2

| | Eg. | | | #F/mol | Yield | % | % |
|---|---|---|---|---|---|---|---|
| Entry | TT | Solvent | i (mA) | alkene | (%) | bis | Z |
| 1 | 2 | 1:1 MeCN:PhCN | 3.7 | 3 | 67 | 37 | 37 |
| 2 | 2 | 1:1 MeCN:PhCN | 3.7 | 3, stir | 38 | 26 | 28 |
| | | | | overnight | | | |
| 3 | 4 | MeCN | 3.7 | 3, stir | 42 | 52 | 50 |
| | | | | overnight | | | |
| 4 | 2 | 1:1 MeCN:PhCN | 7.4 | 2.5 | 46 | 50 | 49 |
| 5 | 2 | 1:1 MeCN:PhCN | 7.4 | 3 | 55 | 33 | 33 |

Table 2 shows, Electrochemical reaction parameters and relevant data for dicationic adduct elimination study. A strong positive correlation (R2=0.99) between the electrogenerated bis:mono ratio and the corresponding Z:E ratio achieved upon adduct elimination provides evidence for a Z-selective bis-adduct elimination.

### Preparation of Pure Bis-Adduct:

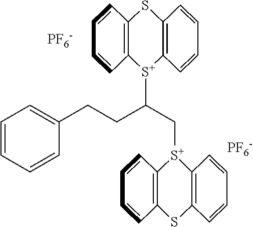

5,5′-(4-Phenylbutane-1,2-diyl)bis(5H-thianthren-5-ium) hexafluorophosphate (bis): Duplicate electrolyses were run side by side and the anolytes combined for ease of isolation. To an oven-dried, divided electrochemical cell equipped with rice grain stir bars were added KPF₆ (295 mg, 1.60 mmol, 4.0 eq.) to both compartments and thianthrene (346 mg, 1.60 mmol, 4.0 eq.) to the anode compartment. The cell was equipped with two septa containing a pencil/RVC anode assembly (electrode dimensions: 0.5×0.5×0.5 cm) and a stainless-steel wire/Ni foam cathode assembly (electrode dimensions: 1×1 cm, folded in half) connected by Teflon tubing to equalize pressure. The cell was flushed with N₂, then each compartment was charged with anhydrous MeCN (4.0 mL) and anhydrous PhCN (4.0 mL). The anode compartment was charged with 4-phenylbutene (60 μL, 0.40 mmol) and trifluoroacetic acid (61 μL, 0.8 mmol, 2.0 eq.), and the cathode compartment was charged with trifluoroacetic acid (153 μL, 2.0 mmol, 5.0 eq.). Banana leads from the power supply were connected to the electrodes, and the contents of the flask were electrolyzed at a constant current of 3.7 mA (J=1.8 mA/cm2) with vigorous stirring at room temperature, during which time an intense purple color developed in the anode compartment. At completion of electrolysis, TFA (1.0 mL) was added, and the combined anolytes were concentrated under reduced pressure. To the purple residue was added diethyl ether (50 mL), swirled vigorously, and then placed in a freezer at −20° C. for 3 h. The resulting mixture was filtered in vacuo. The dark solids were washed with cold diethyl ether, then redissolved in DCM (50 mL). The resulting mixture was filtered in vacuo, and the dark solids were washed with DCM. The purple filtrate was concentrated under reduced pressure to afford the title compound as a foamy white solid (234 mg, 34% yield). ¹H-NMR (500 MHz, CD₃CN) δ 8.16 (d, J=8.0 Hz, 1H), 8.11 (dt, J=8.0, 1.5 Hz, 2H), 8.01 (dd, J=8.4, 7.0 Hz, 1H), 7.98-7.85 (m, 6H), 7.84-7.73 (m, 5H), 7.73-7.64 (m, 1H), 7.56 (t, J=7.7 Hz, 1H), 7.43-7.32 (m, 3H), 7.01 (d, J=7.2 Hz, 2H), 4.20-4.11 (m, 1H), 4.06 (dd, J=13.5, 2.4 Hz, 1H), 3.87 (dd, J=13.5, 9.8 Hz, 1H), 2.74-2.57 (m, 2H), 2.34-2.24 (m, 1H), 2.24-2.15 (m, 1H). ¹⁹F-NMR (400 M Hz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]2+ 282.0531, measured: 282.0529; difference=0.7 ppm). Note: bis decomposes to provide mono and thianthrene overnight in both solution and solid phases, precluding straightforward characterization by ¹³C-NMR. After verifying the purity of the bis-adduct, sodium trifluoroacetate (10 mg) was added directly to the NMR tube, and the contents shaken vigorously before collecting a quantitative ¹H-NMR spectrum (10-second relaxation delay between pulses).

Bis-to-mono conversion studies. An oven-dried, 5-mL vial equipped with a rice grain stir bar was charged with pure bis-adduct (62 mg, 0.072 mmol), MeCN (1.0 mL), and dibromomethane (20.0 μL, 0.288 mmol, 4.0 eq) as an internal standard. The vial was capped, and the contents were stirred vigorously at room temperature. 20-μL aliquots of the reaction were removed and diluted with CD₃CN in an NMR tube for quantitative 1H-NMR analysis (10-second relaxation delay between pulses).

### Substrate Preparation:

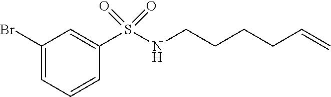 [SMILES: C=CCCCCNS(=O)(=O)c1cccc(Br)c1]

3-Bromo-N-(hex-5-en-1-yl)benzenesulfonamide: To a flame-dried, three-necked, 50-mL round bottom flask equipped with a football stir bar were added potassium carbonate (1.382 g, 10.00 mmol, 2.0 eq.) and 3-bromobenzenesulfonamide (2.361 g, 10.00 mmol, 2.0 eq.). The flask was fitted with a reflux condenser, capped with rubber septa, placed under N₂ atmosphere, then charged sequentially with anhydrous MeCN (20.0 mL) and 6-bromo-1-hexene (815.3 mg, 668 μL, 5.00 mmol). The resulting suspension was stirred vigorously at 105° C. in a bead bath for 18 hours, during which time a yellow reaction color developed. The reaction mixture was cooled to rt, then filtered through a short pad of celite. The filtrate was concentrated under reduced pressure to afford an oily yellow residue which was purified by flash chromatography (10% ethyl acetate in hexanes) to provide the title compound as a crystalline white solid (1.112 g, 70% yield). ¹H-NMR (500 MHz, CD₃CN) 7.97 (t, J=1.9 Hz, 1H), 7.83-7.74 (m, 2H), 7.49 (t, J=7.9 Hz, 1H), 5.76 (ddt, J=16.9, 10.2, 6.7 Hz, 1H), 5.04-4.84 (m, 2H), 2.86 (td, J=6.9, 6.1 Hz, 2H), 2.02-1.95 (m, 2H), 1.47-1.28 (m, 4H). ¹³C-NMR (125 MHz, CD₃CN) 143.54, 139.56, 136.36, 132.08, 130.45, 126.65, 123.38, 115.13, 43.78, 33.74, 29.53, 26.42. HRMS (ESI+) calc: [M−H]− 316.0012, measured: 316.0015; difference=0.9 ppm.

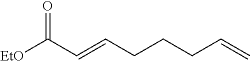

Ethyl (E)-octa-2,7-dienoate: Under N₂ atmosphere, a flame-dried, three-necked, 250-mL roundbottom flask equipped with a football stir bar was charged with anhydrous DCM (35.0 mL) and oxalyl chloride (963 μL, 11.0 mmol, 1.1 eq.). The resulting solution was cooled to −78° C., then charged with DMSO (1.70 mL, 24.0 mmol, 2.4 eq.). After stirring for 10 minutes at −78° C., the flask was charged with hex-5-en-1-ol (1.00 g, 10.0 mmol, 1.0 eq.) dropwise via syringe over 5 minutes. After stirring for 15 minutes at −78° C., the flask was charged with triethylamine (9.62 mL, 69.0 mmol, 6.9 eq.) and stirred for an additional 30 minutes at −78° C., during which time a white precipitate developed. The reaction mixture was warmed to room temperature, and a separately prepared solution of ethyl 2-(triphenyl-15-phosphaneylidene)acetate (5.23 g, 15.0 mmol, 1.5 eq.) in anhydrous DCM (7.9 mL) was added. The resulting solution was stirred at room temperature for 2 hours. Water (25 mL) was added directly to the reaction mixture, and the layers were separated. The aqueous layer was extracted with DCM (3×20 mL), and the combined organic layers were washed with brine, dried over sodium sulfate, and concentrated under reduced pressure to afford a yellow residue which was purified by flash chromatography (10% ethyl acetate in hexanes) to provide the title compound as a clear oil (1.144 g, 68% yield).

¹H-NMR (500 MHz, CDCl₃) δ 6.95 (dt, J=15.6, 7.0 Hz, 1H), 5.85-5.71 (m, 2H), 5.01 (dt, J=17.1, 1.7 Hz, 1H), 4.97 (d, J=10.1 Hz, 1H), 4.18 (q, J=7.1 Hz, 2H), 2.24-2.17 (m, 2H), 2.08 (q, J=7.4 Hz, 2H), 1.56 (p, J=7.5 Hz, 2H), 1.28 (t, J=7.1 Hz, 3H) (consistent with reported spectra: Martin et al. J. Org. Chem. 2005, 70, 10099-10101).

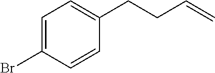

1-Bromo-4-(but-3-en-1-yl)benzene: Adapted from a protocol developed by Molander and coworkers (Molander, G. et al. J. Am. Chem. Soc. 2008, 130, 15792-15793). To a flame-dried 250-mL round-bottom flask equipped with a football stir bar was added 4-bromobenzyl bromide (5.0 g, 20.0 mmol, 1.0 eq.). The flask was capped with a rubber septum, placed under N₂ atmosphere, charged with anhydrous THF (50.0 mL), and cooled to −78° C. with stirring. Next, the flask was charged with allylmagnesium bromide (1.0M in diethyl ether, 22.0 mL, 22.0 mmol, 1.1 eq.) dropwise over 10 minutes, and the resulting solution was stirred at −78° C. for one hour before warming to room temperature and stirring for an additional hour. Water (20 mL) was added directly to the reaction mixture, and the layers were separated. The aqueous layer was extracted with diethyl ether (3×20 mL), and the combined organic layers were washed with brine (80 mL), dried over sodium sulfate, and concentrated under reduced pressure to afford a cloudy oil which was purified by flash chromatography (hexanes) to provide the title compound as a clear oil (3.930 g, 93% yield). ¹H-NMR (500 MHz, CD₃CN) δ 7.43 (dd, J=8.4, 1.0 Hz, 2H), 7.20-7.11 (m, 2H) 5.84 (ddt, J=16.9, 10.2, 6.6 Hz, 1H), 5.02 (dd, J=17.2, 1.9 Hz, 1H), 4.98-4.93 (m, 1H), 2.67 (t, J=7.7 Hz, 2H), 2.39-2.30 (m, 2H), consistent with reported spectra.

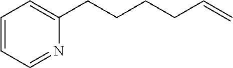

2-(Hex-5-en-1-yl)pyridine: Adapted from a protocol developed by Jiang and coworkers (Jiang, Z. et al. Org. Lett. 2021, 23, 8744-8749). A flame-dried, 100-mL round bottom flask was charged with anhydrous THF (30.0 mL) and 2-methylpyridine (889 μL, 9.0 mmol, 1.0 equiv), and the resulting solution was cooled to −78° C. n-BuLi in hexanes (3.60 mL, 2.50 M, 9.0 mmol, 1.0 equiv) was then added dropwise. The solution was stirred at −78° C. for 30 min, then charged with 5-bromopent-1-ene (1.17 mL, 1.1 equiv, 9.9 mmol) dropwise via syringe. The reaction mixture was stirred at −78° C. for 5 min and then warmed to room temperature. After 1 h, the reaction was quenched with saturated NH4Cl (30 mL) and extracted with ethyl acetate (3×30 mL). The combined organic phases were dried over sodium sulfate and concentrated under reduced pressure to afford a dark yellow oil which was purified by flash chromatography (30% ethyl acetate in hexanes) to provide the title compound as an off-white oil (1.17 g, 81% yield). ¹H-NMR (500 MHz, CD₃CN) δ 8.46 (ddd, J=4.9, 1.9, 1.0 Hz, 1H), 7.63 (td, J=7.7, 1.9 Hz, 1H), 7.19 (d, J=7.8 Hz, 1H), 7.12 (ddd, J=7.6, 4.8, 1.2 Hz, 1H), 5.83 (ddt, J=17.0, 10.2, 6.7 Hz, 1H), 5.00 (dq, J=17.2, 1.7 Hz, 1H), 4.96-4.90 (m, 1H), 2.80-2.72 (m, 2H), 2.14-2.04 (m, 2H), 1.75-1.65 (m, 2H), 1.47-1.38 (m, 2H), consistent with reported spectra.

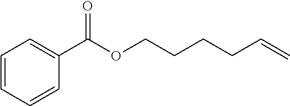

Hex-5-en-1-yl benzoate: Adapted from a protocol developed by Lv and coworkers (Lv, T. et al. J. Fluorine Chem. 2019, 222, 1-7). To an oven-dried, 100-mL round-bottom flask equipped with a football stir bar were added benzoic acid (733 mg, 6.0 mmol, 1.2 eq.), EDCI (1.92 g, 10.0 mmol, 2.0 eq.), and DMAP (122 mg, 1.0 mmol, 0.2 eq.). The flask was fitted with a rubber septum, placed under N₂ atmosphere, and charged with anhydrous DCM (25.0 mL) via syringe. At 0° C., hex-5-en-1-ol (600 μL, 5.0 mmol, 1.0 eq.) was added dropwise via syringe over one minute, and the resulting reaction mixture was allowed to stir at 0° C. for 20 minutes. The ice bath was removed, and stirring was continued for 24 h at room temperature. The reaction mixture was filtered through a short pad of Celite, and the filtrate was concentrated under reduced pressure to afford a yellow oil which was purified by flash chromatography (5% ethyl acetate in hexanes) to provide the title compound as a clear, refractive oil (951 mg, 93% yield). ¹H-NMR (500 MHz, CDCl₃) δ 8.05 (dd, J=8.3, 1.4 Hz, 2H), 7.58-7.52 (m, 1H), 7.44 (dd, J=8.4, 7.2 Hz, 2H), 5.83 (ddt, J=16.9, 10.2, 6.7 Hz, 1H), 5.04 (dd, J=17.1, 1.8 Hz, 1H), 5.00-4.96 (m, 1H), 4.33 (t, J=6.6 Hz, 2H), 2.14 (q, J=7.4 Hz, 2H), 1.84-1.75 (m, 2H), 1.56 (tt, J=9.9, 6.5 Hz, 2H), consistent with reported spectra.

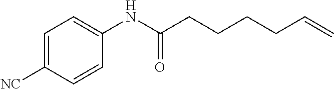 [SMILES: C=CCCCCC(=O)Nc1ccc(C#N)cc1]

N-(4-cyanophenyl)hept-6-enamide: To an oven-dried, 100-mL round-bottom flask equipped with a football stir bar were added EDCI (1.28 g, 6.67 mmol, 1.2 eq.), and DMAP (81.4 mg, 0.67 mmol, 0.12 eq.). The flask was fitted with a rubber septum, placed under N₂ atmosphere, and charged with anhydrous DCM (25.0 mL) via syringe. At 0° C., hept-6-enoic acid (753 μL, 5.56 mmol, 1.0 eq.) was added. After five minutes of stirring, 4-aminobenzonitrile (591 mg, 5.00 mmol, 0.9 eq.) was added dropwise via syringe over one minute and the resulting reaction mixture was allowed to stir at 0° C. for 20 minutes. The ice bath was removed, and stirring was continued for 16 h at room temperature. The reaction was quenched with HCl (1 M, 25 mL) and the organic layer was removed. The aqueous layer was extracted with DCM (2×25 mL). The organic layers were combined, dried over sodium sulfate, and concentrated under reduced pressure to afford a yellow oil which was purified by flash chromatography (15% ethyl acetate in hexanes) to provide the title compound as a white solid (1.01 g, 80% yield). ¹H-NMR (500 MHz, CD₃CN) δ 8.55 (br s, 1H), 7.72 (d, J=8.8 Hz, 2H), 7.64 (d, J=8.7 Hz, 2H), 5.84 (ddt, J=17.0, 10.4, 6.8 Hz, 1H), 5.02 (dt, J=16.6, 1.8 Hz, 1H), 4.95 (d, J=10.3 Hz, 1H), 2.35 (t, J=7.5 Hz, 2H), 2.14-2.02 (m, 2H), 1.66 (p, J=7.6 Hz, 2H), 1.43 (dq, J=10.3, 7.4 Hz, 2H). ¹³C-NMR (126 MHz, CD₃CN) δ 173.13, 144.23, 139.75, 134.15, 120.06, 119.88, 115.05, 106.76, 37.58, 34.10, 29.11, 25.45. HRMS (ESI+) calc: [M+H]+ 229.1335, measured: 229.1331; difference=1.7 ppm.

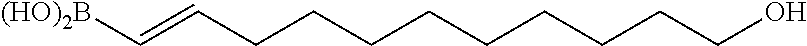 [SMILES: OCCCCCCCCC/C=C/B(O)O]

(E)-(11-hydroxyundec-1-en-1-yl)boronic acid: Adapted from a protocol developed by McElfresh and coworkers (McElfresh, J. et al. J. Chem. Ecology 1999, 25, 687-709). Under N₂ atmosphere, a flame-dried, two-necked, 10-mL round-bottom flask equipped with a rice-grain stir bar was charged with anhydrous THF (1.35 mL) and undec-10-yn-1-ol (673 mg, 4.0 mmol) via syringe. Catecholborane (942 μL, 8.8 mmol, 2.2 eq.) was then added dropwise via syringe. After hydrogen evolution ceased, the mixture was heated to reflux for 20 h. After cooling to rt, the reaction solution was poured into 40 mL of water and stirred for 30 minutes to hydrolyze the boronic ester. The resulting white slurry was cooled in an ice water bath, filtered in vacuo, and the solids were rinsed with cold water (3×20 mL) and then benzene (3×20 mL). The crude product was then extracted with ethyl acetate (3×40 mL), and the combined organic layers were dried over sodium sulfate and concentrated under reduced pressure to afford a thick, off-white oil which crystallized independently to provide the title compound as a sticky white solid (466 mg, 40% yield). ¹H-NMR (500 MHz, MeOD4) δ 6.53 (dt, J=17.6, 6.6 Hz, 1H), 5.56 (d, J=17.6 Hz, 1H), 3.54 (t, J=6.7 Hz, 2H), 2.15 (q, J=7.4 Hz, 2H), 1.59-1.25 (m, 14H). ¹³C-NMR (126 MHz, MeOD4) δ 153.96, 63.01, 36.95, 33.67, 30.69, 30.58, 30.54, 30.29, 29.73, 26.94. Note: the ¹³C-NMR resonance for the alkenyl carbon bonded to boron is missing due to peak broadening and splitting from ¹¹B coupling. ¹¹B-NMR (128 MHz, MeOD4) δ 27.4. HRMS (ASAP) calc: [M+H]+ 215.1813, measured: 215.1812; difference=0.5 ppm.

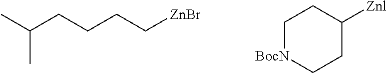

Alkylzinc reagents were prepared according to a protocol developed by Harrity and coworkers (Harrity, J. et al. Eur. J. Inorg. Chem. 2016, 83-86). A flame-dried, three-necked, 50-mL roundbottom flask equipped with a football stir bar and reflux condenser was charged with zinc dust (1.308 g, 20.0 mmol, 2.0 eq.) and oven-dried LiCl (848 mg, 20.0 mmol, 2.0 eq.). The flask was capped with rubber septa, placed under an N₂ atmosphere, and charged with anhydrous THF (9.0 mL). 1,2-dibromoethane (100 μL, 1.16 mmol, 0.12 eq.) was added, and the reaction mixture was heated to reflux with a heat gun with stirring. This process was repeated further two times. The reaction mixture was cooled to rt, and TMSCl (100 μL, 0.79 mmol, 0.08 eq.) was added slowly, followed by 10 minutes of stirring. A solution of iodine (127 mg, 0.50 mmol, 0.05 eq.) in THF (1.00 mL) was added, and the reaction mixture was stirred until the resulting brown color had dissipated (around 2 minutes). Alkyl iodide (10.00 mmol) was added neat (solid alkyl iodides were dissolved in 2.0 mL of THF prior to addition), and the resulting reaction mixture was stirred vigorously at 50° C. for 18 h. The molarity of the afforded alkylzinc iodides was determined by iodometric titration in a 0.5 M solution of LiCl in THF using a protocol developed by Knochel and coworkers (Knochel, P. et al. Synthesis 2006, 5, 890-891).

### General Cross-Coupling Procedures:

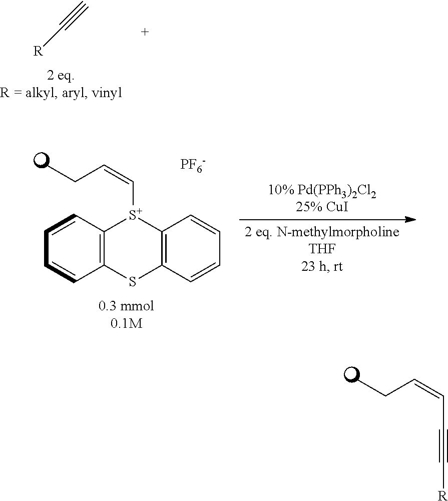

General Procedure B: C(sp2)-C(sp) Sonogashira Coupling: In an N₂-filled glovebox, to a 1-dram borosilicate vial equipped with a rice-grain stir bar were added Pd(PPh₃)₂Cl₂ (21 mg, 0.03 mmol, 0.1 eq.), CuI (14 mg, 0.075 mmol, 0.25 eq.), and alkenyl thianthrenium salt (0.3 mmol). The vial was charged sequentially with anhydrous THF (3.0 mL), N-methylmorpholine (66 μL, 0.6 mmol, 2.0 eq.), and alkyne coupling partner (0.6 mmol, 2.0 eq.), fitted with a Teflon screw cap, and allowed to stir at room temperature for 23 h. At completion of reaction, the vial was removed from the glovebox, and the contents were filtered through a short pad of Celite, eluting with ethyl acetate. The filtrate was concentrated under reduced pressure and the residue purified by flash column chromatography to afford the pure Sonogashira product.

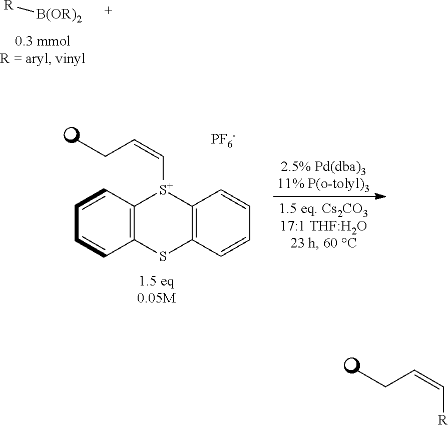

General Procedure C: C(sp2)-C(sp2) Suzuki Coupling: In an N₂-filled glovebox, to a 2-dram borosilicate vial equipped with a rice-grain stir bar were added Pd₂(dba)₃ (6.9 mg, 0.0075 mmol, 0.025 eq.), P(o-tolyl)₃ (10 mg, 0.033 mmol, 0.11 eq.), Cs₂CO₃ (147 mg, 0.45 mmol, 1.5 eq.), boronic ester or acid coupling partner (0.3 mmol), and Z-alkenyl thianthrenium salt (0.45 mmol, 1.5 eq.). The vial was charged with anhydrous THF (6.0 mL), fitted with a septum screw cap, removed from the glovebox, and charged with degassed, deionized water (540 μL) via syringe. The resulting mixture was stirred vigorously in a heating block at 60° C. for 23 h. At completion of reaction, the contents of the vial were cooled to room temperature and filtered through a short pad of Celite, eluting with ethyl acetate. The filtrate was concentrated under reduced pressure and the residue purified by flash column chromatography to afford the pure Suzuki product.

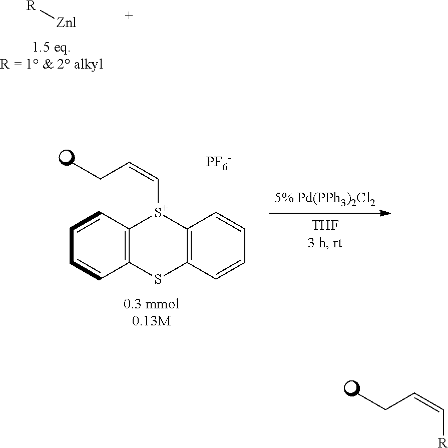

General Procedure D: C(sp2)-C(sp3) Negishi Coupling: In an N₂-filled glovebox, to a 1-dram borosilicate vial equipped with a rice-grain stir bar were added Pd(PPh₃)₂Cl₂ (10.5 mg, 0.015 mmol, 0.05 eq.) and Z-alkenyl thianthrenium salt (0.3 mmol). The vial was charged with anhydrous THF (2.25 mL) and 1° or 2° alkylzinc iodide coupling partner (0.45 mmol, 1.5 eq.), fitted with a Teflon screw cap, and allowed to stir at room temperature for 3 h. At completion of reaction, the vial was removed from the glovebox, and the contents were filtered through a short pad of Celite, eluting with ethyl acetate. The filtrate was concentrated under reduced pressure and the residue purified by flash column chromatography to afford the pure Negishi product.

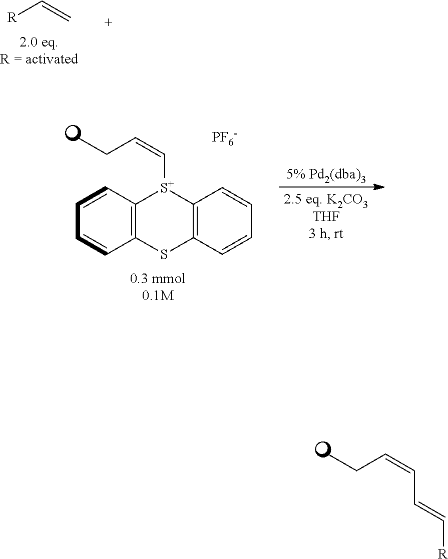

General Procedure E: C(sp2)-C(sp2) Heck Reaction: In an N₂-filled glovebox, to a 2-dram borosilicate vial equipped with a rice-grain stir bar were added Pd2(dba)₃ (13.7 mg, 0.015 mmol, 0.05 eq.), potassium carbonate (104 mg, 0.75 mmol, 2.5 eq.), and Z-alkenyl thianthrenium salt (0.30 mmol). The vial was charged with anhydrous THF (3.0 mL) and alkene coupling partner (0.6 mmol, 2.0 eq.), fitted with a Teflon screw cap, and allowed to stir at room temperature for 3 h. At completion of reaction, the vial was removed from the glovebox, and the contents were filtered through a short pad of Celite, eluting with ethyl acetate. The filtrate was concentrated under reduced pressure and the residue purified by flash column chromatography to afford the pure Heck product.

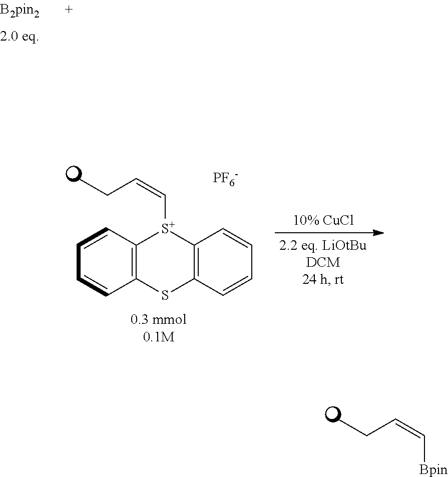

General Procedure F: C(sp2) Borylation Reaction: In an N₂-filled glovebox, to a 2-dram borosilicate vial equipped with a rice-grain stir bar were added CuCl (2.9 mg, 0.03 mmol, 0.10 eq.), lithium tert-butoxide (53 mg, 0.66 mmol, 2.2 eq.), B₂pin₂ (152 mg, 0.60 mmol, 2.0 eq.), and Z-alkenyl thianthrenium salt (0.30 mmol). The vial was charged with anhydrous DCM (3.0 mL), fitted with a Teflon screw cap, and allowed to stir at room temperature for 24 h. At completion of reaction, the vial was removed from the glovebox, and the contents were filtered through a short pad of Celite, eluting with DCM. The filtrate was concentrated under reduced pressure and the residue purified by flash column chromatography to afford the pure alkenyl boronic ester product.

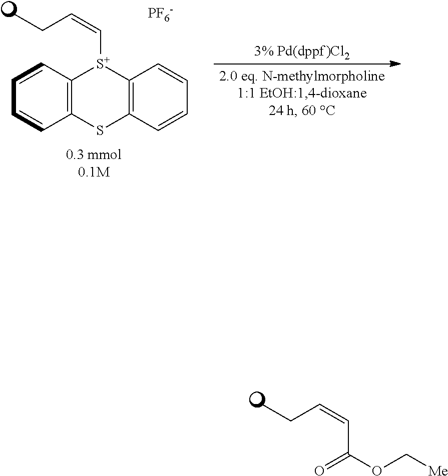

General Procedure G: C(sp2) Carbonylation Reaction: Under ambient atmosphere, to an oven dried 25-mL round-bottom flask equipped with a rice-grain stir bar were added Pd(dppf)Cl₂ (11 mg, 0.015 mmol, 0.03 eq.) and Z-alkenyl thianthrenium salt (0.30 mmol). The flask was fitted with a rubber septum and charged with ethanol (2.0 mL) and 1,4-dioxane (2.0 mL) via syringe. The resulting solution was sparged first with N₂ for 5 minutes and then with CO for 5 minutes. To the flask was added N-methylmorpholine (110 μL, 1.0 mmol, 2.0 eq.) via syringe, and the reaction mixture was allowed to stir at 60° C. for 24 h. At completion of reaction, the reaction mixture was extracted with ethyl acetate (3×20 mL). The combined organic extracts were dried over Na₂SO₄ and concentrated under reduced pressure. The residue was purified by flash column chromatography to afford the pure carbonylation product.

### Evidence for Aryl Bromide Retention in Catalytic Cross-Couplings:

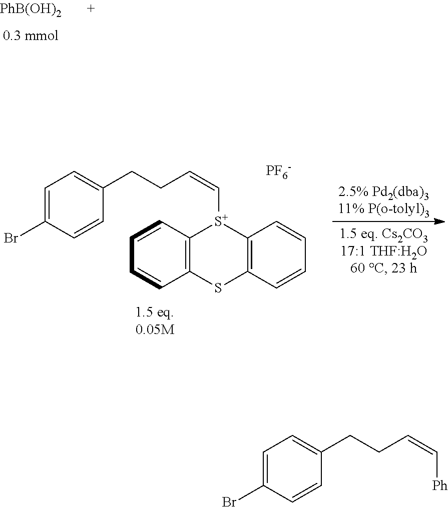

(Z)-5-(4-(4-bromophenyl)but-1-en-1-yl)-5H-thianthren-5-ium hexafluorophosphate was coupled to PhB(OH)₂ via General Procedure C (78% crude yield). GCMS analysis of the worked up crude material indicated full retention of the aryl bromide. (Data not shown.)

### Product Isolation and Characterization:

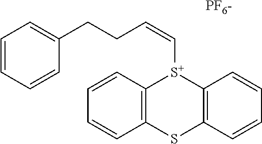 [SMILES: C(=C/[S+]1c2ccccc2Sc2ccccc21)/CCc1ccccc1 ; "PF6-"]

(Z)-5-(4-phenylbut-1-en-1-yl)-5H-thianthren-5-ium hexafluorophosphate (1): Prepared according to General Procedure A. Purified by recrystallization (DCM/ethyl acetate). Morphology: crystalline white solid. Purified yield: 1.047 g, 69%, >20:1 Z:E. ¹H-NMR (500 MHz, CD₃CN) 7.92 (dd, J=7.95, 1.26 Hz, 2H), 7.82 (dd, J=7.97, 1.32 Hz, 2H), 7.76 (td, J=7.70, 1.37 Hz, 2H), 7.65 (td, J=7.73, 1.29 Hz, 2H), 7.32-7.21 (m, 5H), 6.90 (dt, J=8.66, 7.83 Hz, 1H), 6.69 (dt, J=8.58, 1.36 Hz, 1H), 2.99 (m, 2H), 2.86 (t, J=7.01 Hz, 2H). ¹³C-NMR (125 MHz, CD₃CN) 155.84, 140.91, 136.22, 135.28, 133.04, 131.52, 130.97, 129.72, 129.70, 127.63, 122.07, 112.84, 34.26, 33.04. ¹⁹F-NMR (400 MHz, CD₃CN) −72.85 (d, J=706.59 Hz, 6F). ³¹P-NMR (400 MHz, CD₃CN) −144.61 (hept, J=706.56 Hz, 1P). HRMS (ESI+) calc: [M]+ 347.0923, measured: 347.0927; difference=1.2 ppm.

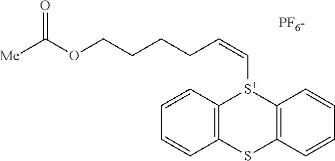 [SMILES: CC(=O)OCCCC/C=C\[S+]1c2ccccc2Sc2ccccc21 ; "PF6-"]

(Z)-5-(6-acetoxyhex-1-en-1-yl)-5H-thianthren-5-ium hexafluorophosphate (3): Prepared according to General Procedure A. Purified by recrystallization (DCM/ethyl acetate). Morphology: crystalline off-white solid. Purified yield: 1.127 g, 72%, >20:1 Z:E. ¹H-NMR (500 MHz, CD₃CN) 8.12 (dd, J=7.97, 1.34 Hz, 2H), 7.97 (dd, J=7.96, 1.25 Hz, 2H), 7.81 (td, J=7.70, 1.37 Hz, 2H), 7.71 (td, J=7.75, 1.33 Hz, 2H), 6.82 (dt, J=8.59, 7.73 Hz, 1H), 6.69 (dt, J=8.56, 1.37 Hz, 1H), 4.04 (t, J=6.50, 2H), 2.67 (qd, J=7.57, 1.37 Hz), 1.99 (s, 3H), 1.64 (m, 2H), 1.49 (m, 2H). ¹³C-NMR (125 MHz, CD₃CN) 171.77, 155.95, 136.69, 135.48, 133.47, 131.70, 131.02, 122.23, 113.05, 64.44, 30.81, 28.86, 25.25, 21.15. ¹⁹F-NMR (400 MHz, CD₃CN) −71.83 (d, J=706.40 Hz, 6F). ³¹P-NMR (400 MHz, CD₃CN) −144.63 (hept, J=706.43 Hz, 1P). HRMS (ESI+) calc: [M]+ 357.0978, measured: 357.0973; difference=1.4 ppm.

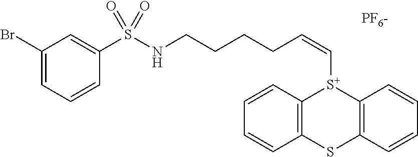 [SMILES: O=S(=O)(NCCCC/C=C\[S+]1c2ccccc2Sc2ccccc21)c1cccc(Br)c1 ; "PF6-"]

(Z)-5-(6-((3-bromophenyl)sulfonamido)hex-1-en-1-yl)-5H-thianthren-5-ium hexafluorophosphate (4): Prepared according to General Procedure A. Purified by precipitation from DCM. Morphology: powdery off-white solid. Purified yield: 1.410 g, 67%, >20:1 Z:E. ¹H-NMR (500 MHz, CD₃CN) 8.12 (dd, J=8.0, 1.3 Hz, 2H), 8.02-7.93 (m, 3H), 7.84-7.76 (m, 4H), 7.71 (td, J=7.8, 1.3 Hz, 2H), 7.50 (t, J=7.9 Hz, 1H), 6.77 (q, J=7.9 Hz, 1H), 6.67 (dt, J=8.5, 1.4 Hz, 1H), 5.66 (t, J=6.3 Hz, 1H), 2.87 (q, J=6.2 Hz, 2H), 2.64-2.57 (m, 2H), 1.45 (dd, J=4.7, 1.9 Hz, 4H). ¹³C-NMR (126 MHz, CD₃CN) δ 155.78, 143.29, 136.59, 136.48, 135.41, 133.40, 132.16, 131.60, 130.98, 130.42, 126.63, 123.41, 122.08, 112.95, 43.37, 30.54, 29.50, 25.62. ¹⁹F-NMR (400 MHz, CD₃CN) −71.83 (d, J=706.40 Hz, 6F). ³¹P-NMR (400 MHz, CD₃CN) −144.63 (hept, J=706.43 Hz, 1P). HRMS (ESI+) calc: [M]+ 532.0069, measured: 532.0071; difference=0.4 ppm.

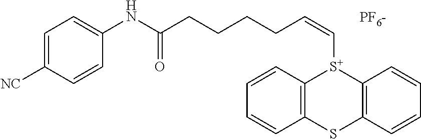

(Z)-5-(7-((4-cyanophenyl)amino-7-oxohept-1-en-1-yl)-5H-thianthren-5-ium hexafluorophosphate (5): Prepared according to General Procedure A. Purified by recrystallization (DCM/ethyl acetate). Morphology: crystalline white solid. Purified yield: 1.16 g, 63%, >20:1 Z:E. ¹H-NMR (500 MHz, CD₃CN) δ 8.57 (br s, 1H), 8.12 (dd, J=8.0, 1.3 Hz, 2H), 7.96 (dd, J=7.9, 1.3 Hz, 2H), 7.78 (td, J=7.7, 1.3 Hz, 2H), 7.73 (d, J=8.8 Hz, 2H), 7.70-7.64 (m, 4H), 6.85 (q, J=8.0 Hz, 1H), 6.70 (d, J=8.5 Hz, 1H), 2.68 (qd, J=7.5, 1.4 Hz, 2H), 2.38 (t, J=7.2 Hz, 2H), 1.74-1.65 (m, 2H), 1.51 (tt, J=10.4, 6.3 Hz, 2H). ¹³C-NMR (126 MHz, CD₃CN) δ 172.73, 156.09, 144.06, 136.48, 135.34, 134.16, 133.32, 131.54, 130.93, 122.10, 120.07, 119.84, 112.81, 106.83, 37.13, 30.92, 28.15, 25.23. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 443.1246, measured: 443.1239; difference=1.6 ppm.

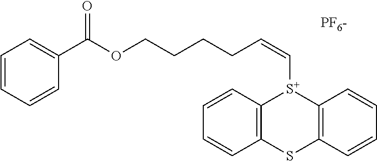 [SMILES: O=C(OCCCC/C=C\[S+]1c2ccccc2Sc2ccccc21)c1ccccc1 ; "PF6-"]

(Z)-5-(6-benzoyloxyhex-1-en-1-yl)-5H-thianthren-5-ium hexafluorophosphate (6): Prepared according to General Procedure A. Purified by recrystallization (hot ethanol, then DCM/ethanol). Morphology: crystalline off-white solid. Purified yield: 1.09 g, 63%, >20:1 Z:E. ¹H-NMR (500 MHz, CD₃CN) δ 8.10 (dd, J=8.0, 1.4 Hz, 2H), 8.00 (dd, J=8.3, 1.4 Hz, 2H), 7.94 (dd, J=7.9, 1.3 Hz, 2H), 7.78 (td, J=7.7, 1.3 Hz, 2H), 7.67 (td, J=7.7, 1.3 Hz, 2H), 7.65-7.60 (m, 1H), 7.53-7.47 (m, 2H), 6.86 (dt, J=8.5, 7.7 Hz, 1H), 6.69 (d, J=8.6 Hz, 1H), 4.32 (t, J=6.4 Hz, 2H), 2.72 (qd, J=7.6, 1.4 Hz, 2H), 1.80 (dq, J=8.3, 6.5 Hz, 2H), 1.61 (tt, J=9.9, 6.4 Hz, 2H). ¹³CNMR (126 MHz, CD₃CN) δ 167.15, 155.99, 136.53, 135.35, 134.05, 133.30, 131.57, 131.36, 130.92, 130.18, 129.56, 122.12, 118.26, 112.90, 65.11, 30.75, 28.83, 25.31, 1.25. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 419.1134, measured: 419.1133; difference=0.2 ppm.

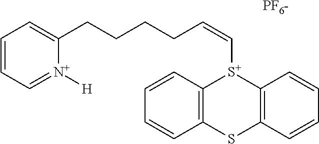 [SMILES: C(=C/[S+]1c2ccccc2Sc2ccccc21)/CCCCc1cccc[nH+]1 ; "PF6-"]

(Z)-2-(6-(5H-thianthren-5-ium-5-yl)hex-5-en-1-yl)pyridin-1-ium hexafluorophosphate (7): Prepared according to General Procedure A with the following modification: following aqueous workup, the solid crude material was dissolved in DCM and stirred with trifluoroacetic acid (2.37 mL, 31.0 mmol, 10.0 eq.) for 15 min, then concentrated under reduced pressure and purified by trituration from DCM (−20° C.). Morphology: crystalline white solid. Purified yield: 967 mg, 39%, >20:1 Z:E. ¹H-NMR (500 MHz, CD₃CN) δ 8.57-8.53 (m, 1H), 8.50 (td, J=7.8, 1.7 Hz, 1H), 8.10 (dq, J=8.1, 1.3 Hz, 2H), 7.97 (dd, J=7.9, 1.3 Hz, 2H), 7.92-7.83 (m, 2H), 7.80 (td, J=7.7, 1.4 Hz, 2H), 7.69 (td, J=7.7, 1.3 Hz, 2H), 6.81 (q, J=8.0 Hz, 1H), 6.72-6.67 (m, 1H), 3.07-2.95 (m, 2H), 2.68 (q, J=7.4 Hz, 2H), 1.81-1.71 (m, 2H), 1.52 (p, J=7.5 Hz, 2H). ¹³C-NMR (126 MHz, CD₃CN) δ 157.87, 155.37, 148.16, 141.68, 136.44, 135.27, 133.28, 131.44, 130.82, 128.28, 125.91, 121.92, 113.03, 33.55, 30.53, 28.62, 27.69. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 376.1188, measured: 376.1183; difference=1.3 ppm.

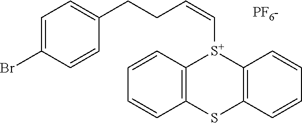

(Z)-5-(4-(4-bromophenyl)but-1-en-1-yl)-5H-thianthren-5-ium hexafluorophosphate (8): Prepared according to General Procedure A. Purified by recrystallization (DCM/ethyl acetate). Morphology: crystalline white solid. Purified yield: 997 mg, 55%, >20:1 Z:E. ¹H-NMR (500 MHz, CD₃CN) 7.92 (dd, J=8.0, 1.3 Hz, 2H), 7.78 (qd, J=7.9, 1.3 Hz, 2H), 7.66 (td, J=7.7, 1.3 Hz, 2H), 7.42 (d, J=8.3 Hz, 2H), 7.14 (d, J=8.4 Hz, 2H), 6.89 (q, J=8.0 Hz, 1H), 6.70 (d, J=8.6 Hz, 1H), 3.05-2.91 (d, J=7.9 Hz, 2H), 2.83 (t, J=6.9 Hz, 2H). ¹³C-NMR (126 MHz, CD₃CN) δ 155.67, 140.18, 136.17, 135.23, 132.81, 132.57, 131.82, 131.52, 130.95, 122.06, 120.88, 112.96, 33.56, 32.82. ¹⁹F-NMR (400 MHz, CD₃CN) −71.83 (d, J=706.40 Hz, 6F). ³¹P-NMR (400 MHz, CD₃CN) −144.63 (hept, J=706.43 Hz, 1P). HRMS (ESI+) calc: [M]+ 425.0028, measured: 425.0026; difference=0.5 ppm.

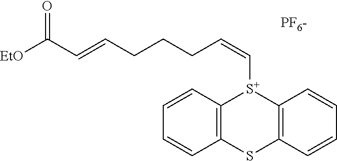 [SMILES: CCOC(=O)/C=C/CCC/C=C\[S+]1c2ccccc2Sc2ccccc21 ; "PF6-"]

5-((1Z,6E)-8-ethoxy-8-oxoocta-1,6-dien-1-yl)-5H-thianthren-5-ium hexafluorophosphate (9): Prepared according to General Procedure A. Purified by flash chromatography (linear gradient of 20-60% acetone in hexanes) followed by recrystallization (warm methanol). Morphology: crystalline white solid. Purified yield: 814 mg, 50%, >20:1 Z:E. ¹H-NMR (500 MHz, CD₃CN) δ 8.11 (dd, J=7.9, 1.4 Hz, 2H), 7.96 (dd, J=8.0, 1.2 Hz, 2H), 7.80 (td, J=7.7, 1.4 Hz, 2H), 7.71 (td, J=7.7, 1.3 Hz, 2H), 6.90 (dt, J=15.6, 6.9 Hz, 1H), 6.82 (dt, J=8.5, 7.7 Hz, 1H), 6.68 (dt, J=8.5, 1.4 Hz, 1H), 5.84 (dt, J=15.7, 1.6 Hz, 1H), 4.15 (q, J=7.1 Hz, 2H), 2.65 (qd, J=7.6, 1.4 Hz, 2H), 2.25 (qd, J=7.1, 1.6 Hz, 2H), 1.61 (p, J=7.5 Hz, 2H), 1.25 (t, J=7.1 Hz, 3H). ¹³CNMR (126 MHz, CD₃CN) δ 167.07, 155.43, 148.87, 136.23, 135.14, 133.10, 131.29, 130.71, 122.53, 121.75, 112.75, 60.81, 31.58, 30.29, 26.71, 14.29. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 383.1134, measured: 383.1128; difference=1.6 ppm.

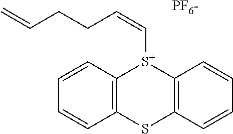 [SMILES: C=CCC/C=C\[S+]1c2ccccc2Sc2ccccc21 ; "PF6-"]

(Z)-5-(hexa-1,5-dien-1-yl)-5H-thianthren-5-ium hexafluorophosphate (10): Prepared according to General Procedure A. Purified by flash chromatography (linear gradient of 0-60% acetone in hexanes) followed by recrystallization (DCM/ethyl acetate). Morphology: crystalline white solid. Purified yield: 752 mg, 55%, >20:1 Z:E. ¹H-NMR (500 MHz, CD₃CN) 8.11 (dd, J=7.9, 1.4 Hz, 2H), 7.97 (dd, J=7.9, 1.4 Hz, 2H), 7.81 (td, J=7.7, 1.4 Hz, 2H), 7.71 (td, J=7.7, 1.3 Hz, 2H), 6.85-6.76 (m, 1H), 6.72 (dt, J=8.7, 1.3 Hz, 1H), 5.80 (ddt, J=17.0, 10.2, 6.7 Hz, 1H), 5.07 (dq, J=17.2, 1.6 Hz, 1H), 5.02 (dq, J=10.1, 1.3 Hz, 1H), 2.75 (qd, J=7.3, 1.4 Hz, 2H), 2.24 (qt, J=7.1, 1.4 Hz, 2H). ¹³C-NMR (125 MHz, CD₃CN) 155.58, 137.33, 136.61, 135.45, 133.48, 131.64, 131.01, 122.09, 117.25, 113.04, 32.49, 30.51. ¹⁹F-NMR (400 MHz, CD₃CN) −72.77 (d, J=706.8 Hz, 6F). ³¹P-NMR (400 MHz, CD₃CN) −144.59 (hept, J=706.7 Hz, 1P). HRMS (ESI+) calc: [M]+ 297.0766, measured: 297.0762; difference=1.3 ppm.

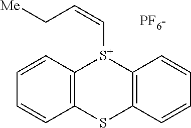

(Z)-5-(but-1-en-1-yl)-5H-thianthren-5-ium hexafluorophosphate (11): Prepared according to General Procedure A with the gaseous alkene modifications. Purified by recrystallization (methanol at −20° C.). Morphology: crystalline white solid. Purified yield: 1.53 g, 59%, >20:1 Z:E. ¹H-NMR (500 MHz, CD₃CN) δ 8.12 (dd, J=8.1, 1.3 Hz, 2H), 7.96 (dd, J=7.9, 1.3 Hz, 2H), 7.80 (td, J=7.7, 1.4 Hz, 2H), 7.71 (td, J=7.7, 1.3 Hz, 2H), 6.82 (q, J=7.9 Hz, 1H), 6.71-6.59 (d, J=8.6 Hz, 1H), 2.66 (p, J=7.5 Hz, 2H), 1.07 (t, J=7.4 Hz, 3H). ¹³C-NMR (126 MHz, CD₃CN) δ 157.53, 136.50, 135.32, 133.31, 131.53, 130.90, 122.15, 112.03, 24.76, 12.75. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 271.0610, measured: 271.0605; difference=1.8 ppm.

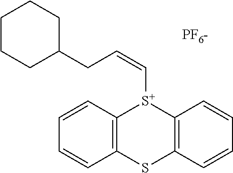 [SMILES: C(=C/[S+]1c2ccccc2Sc2ccccc21)/CC1CCCCC1 ; "PF6-"]

(Z)-5-(3-cyclohexylprop-1-en-1-yl)-5H-thianthren-5-ium hexafluorophosphate (12): Prepared according to General Procedure A. Purified by recrystallization (DCM/ethyl acetate). Morphology: crystalline white solid. Purified yield: 911 mg, 61%, >20:1 Z:E. ¹H-NMR (500 MHz, CD₃CN) 8.11 (dd, J=8.0, 1.3 Hz, 2H), 7.97 (dd, J=7.9, 1.3 Hz, 2H), 7.81 (td, J=7.8, 1.4 Hz, 2H), 7.71 (td, J=7.7, 1.3 Hz, 2H), 6.86 (q, J=8.0 Hz, 1H), 6.72 (d, J=8.7 Hz, 1H), 2.55 (ddd, J=8.0, 6.8, 1.4 Hz, 2H), 1.73-1.60 (m, 5H), 1.53-1.39 (m, 1H), 1.31-1.11 (m, 3H), 1.00 (qd, J=11.7, 2.9 Hz, 2H). ¹³C-NMR (126 MHz, CD₃CN) δ 155.54, 136.61, 135.38, 133.39, 131.61, 130.94, 122.10, 113.03, 38.48, 38.18, 33.48, 26.79, 26.74. ¹⁹F-NMR (400 MHz, CD₃CN) −71.83 (d, J=706.40 Hz, 6F). ³¹P-NMR (400 MHz, CD₃CN) −144.63 (hept, J=706.43 Hz, 1P). HRMS (ESI+) calc: [M]+ 339.1236, measured: 339.1234; difference=0.6 ppm.

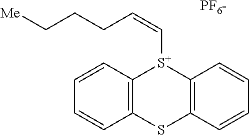 [SMILES: CCCC/C=C\[S+]1c2ccccc2Sc2ccccc21 ; "PF6-"]

(Z)-5-(hex-1-en-1-yl)-5H-thianthren-5-ium hexafluorophosphate (19): Prepared according to General Procedure A with the following modification: after aqueous workup, diethyl ether (50 mL) was added, and the mixture was stored overnight at −20° C., then further cooled to −78° C. in a dry ice/acetone bath for 2 h. The resulting precipitate was filtered in vacuo and then purified by flash chromatography (linear gradient of 0-75% acetone in hexanes). Morphology: crystalline white solid. Purified yield: 828 mg, 60%, >20:1 Z:E. ¹H-NMR (500 MHz, CD₃CN) δ 8.11 (dd, J=8.0, 1.4 Hz, 2H), 7.97 (dd, J=7.9, 1.3 Hz, 2H), 7.81 (td, J=7.7, 1.4 Hz, 2H), 7.71 (td, J=7.7, 1.3 Hz, 2H), 6.84 (q, J=7.9 Hz, 1H), 6.67 (d, J=8.6 Hz, 1H), 2.64 (q, J=7.7 Hz, 1H), 1.48-1.29 (m, 4H), 0.92 (t, J=7.2 Hz, 3H). ¹³C-NMR (126 MHz, CD₃CN) δ 156.49, 136.61, 135.38, 133.36, 131.62, 130.95, 122.21, 112.55, 30.97, 30.77, 22.91, 13.99. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 299.0923, measured: 299.0922; difference=0.3 ppm.

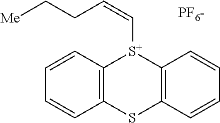

(Z)-5-(pent-1-en-1-yl)-5H-thianthren-5-ium hexafluorophosphate (21): Prepared according to General Procedure A with the following modification: after aqueous workup, diethyl ether (50 mL) was added, and the mixture was stored overnight at −20° C., then further cooled to −78° C. in a dry ice/acetone bath for 2 h. The resulting precipitate was filtered in vacuo and then purified by flash chromatography (linear gradient of 0-75% acetone in hexanes). Morphology: crystalline white solid. Purified yield: 911 mg, 68%, >20:1 Z:E. ¹H-NMR (500 MHz, CD₃CN) δ 8.12 (dd, J=8.0, 1.4 Hz, 2H), 7.95 (dd, J=8.0, 1.3 Hz, 2H), 7.80 (td, J=7.7, 1.4 Hz, 2H), 7.70 (td, J=7.7, 1.3 Hz, 2H), 6.83 (dt, J=8.5, 7.7 Hz, 1H), 6.69 (dt, J=8.6, 1.4 Hz, 1H), 2.63 (qd, J=7.4, 1.4 Hz, 2H), 1.49 (h, J=7.4 Hz, 2H), 0.94 (t, J=7.4 Hz, 3H). ¹³C-NMR (126 MHz, CD₃CN) δ 156.30, 136.52, 135.34, 133.34, 131.54, 130.92, 122.10, 112.71, 32.91, 22.07, 13.72. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 285.0766, measured: 285.0764; difference=0.7 ppm.

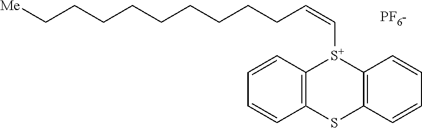

(Z)-5-(dodec-1-en-1-yl)-5H-thianthren-5-ium hexafluorophosphate (24): Prepared according to General Procedure A with the following modification: benzonitrile was removed by short path distillation following aqueous workup of the crude mixture. Purified by flash chromatography (linear gradient of 10-60% acetone in hexanes) followed by recrystallization (methanol at −20° C.). Morphology: crystalline white solid. Purified yield: 1020 mg, 62%, >20:1 Z:E. ¹H-NMR (500 MHz, CD₃CN) δ 8.11 (d, J=7.9 Hz, 2H), 8.00-7.95 (m, 2H), 7.81 (t, J=7.7 Hz, 2H), 7.74-7.67 (m, 2H), 6.84 (q, J=8.0 Hz, 1H), 6.67 (d, J=8.5 Hz, 1H), 2.64 (q, J=7.4 Hz, 1H), 2.14 (s, 1H), 1.44 (t, J=7.2 Hz, 1H), 1.27 (d, J=4.9 Hz, 5H), 0.89 (t, J=6.7 Hz, 1H). ¹³C-NMR (126 MHz, CD₃CN) δ 156.63, 136.60, 135.37, 133.33, 131.62, 130.94, 122.26, 112.53, 32.60, 31.25, 30.21, 30.14, 30.00, 29.95, 29.74, 28.67, 23.36, 14.37. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 383.1862, measured: 383.1859; difference=0.8 ppm

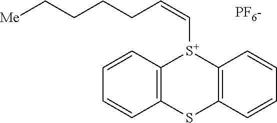

(Z)-5-(hept-1-en-1-yl)-5H-thianthren-5-ium hexafluorophosphate (26): Prepared according to General Procedure A. Purified by flash chromatography (linear gradient of 10-60% acetone in hexanes) followed by trituration in ethyl ether. Morphology: crystalline white solid. Purified yield: 908 mg, 64%, >20:1 Z:E. ¹H-NMR (500 MHz, CD₃CN) δ 8.11 (dd, J=7.9, 1.4 Hz, 2H), 7.97 (dd, J=7.9, 1.3 Hz, 2H), 7.81 (td, J=7.7, 1.4 Hz, 2H), 7.71 (td, J=7.7, 1.3 Hz, 2H), 6.83 (q, J=8.0 Hz, 1H), 6.67 (dt, J=8.5, 1.3 Hz, 1H), 2.64 (qd, J=7.6, 1.3 Hz, 2H), 1.44 (p, J=7.3 Hz, 2H), 1.37-1.22 (m, 4H), 0.89 (t, J=6.8 Hz, 3H). ¹³C-NMR (126 MHz, CD₃CN) δ 156.51, 136.62, 135.39, 133.38, 131.62, 130.94, 122.22, 112.58, 31.94, 31.20, 28.37, 23.03, 14.15. ¹⁹F-NMR (400 MHz, CD₃CN) δ −71.83 (d, J=706.40 Hz, 6F). ³¹P-NMR (400 MHz, CD₃CN) δ −144.63 (hept, J=706.43 Hz, 1P). HRMS (ESI+) calc: [M]+ 313.1079, measured: 313.1077; difference=0.6 ppm.

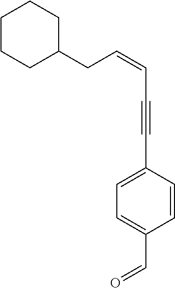 [SMILES: O=Cc1ccc(C#C/C=C\CC2CCCCC2)cc1]

(Z)-4-(5-cyclohexylpent-3-en-1-yn-1-yl)benzaldehyde (13): Prepared according to General Procedure B. Purified by flash chromatography (1% ethyl acetate in hexanes, then linear gradient from 1-5% ethyl acetate in hexanes). Morphology: clear oil. Purified yield: 63 mg, 83%, >20:1 Z:E. Note: compound readily isomerizes to E-isomer upon exposure to sunlight. ¹H-NMR (500 MHz, CDCl₃) δ 9.99 (s, 1H), 7.82 (d, J=8.2 Hz, 2H), 7.55 (d, J=8.2 Hz, 2H), 6.08 (dt, J=10.8, 7.6 Hz, 1H), 5.72 (dt, J=10.9, 1.4 Hz, 1H), 2.31 (td, J=7.3, 1.4 Hz, 2H), 1.73 (tt, J=15.1, 2.1 Hz, 5H), 1.43 (m, 1H), 1.32-1.10 (m, 3H), 1.07-0.95 (m, 2H). ¹³C-NMR (126 MHz, CDCl₃) δ 191.45, 144.90, 135.29, 131.95, 130.19, 129.64, 109.32, 92.59, 91.04, 38.43, 38.03, 33.27, 26.55, 26.39. HRMS (ESI+) calc: [M+H]+ 253.1587, measured: 253.1582; difference=2.0 ppm.

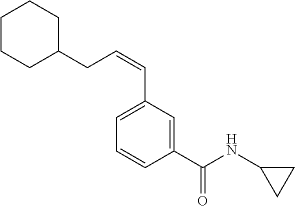 [SMILES: O=C(NC1CC1)c1cccc(/C=C\CC2CCCCC2)c1]

(Z)-3-(3-cyclohexylprop-1-en-1-yl)-N-cyclopropylbenzamide (14): Prepared according to General Procedure C. Purified by flash chromatography (15% ethyl acetate in hexanes). Morphology: clear oil. Purified yield: 60 mg, 71% yield, >20:1 Z:E. ¹H-NMR (500 MHz, CDCl₃) δ 7.62 (d, J=1.8 Hz, 1H), 7.55 (dt, J=7.3, 1.7 Hz, 1H), 7.41-7.32 (m, 2H), 6.44 (d, J=11.7 Hz, 1H), 6.24 (s, 1H), 5.75 (dt, J=11.7, 7.4 Hz, 1H), 2.91 (tq, J=7.1, 3.6 Hz, 1H), 2.19 (td, J=7.1, 1.9 Hz, 2H), 1.79-1.57 (m, 5H), 1.44-1.31 (m, 1H), 1.30-1.06 (m, 3H), 0.97-0.82 (m, 3H), 0.67-0.55 (m, 2H). ¹³C-NMR (126 MHz, CDCl₃) δ 169.11, 138.36, 134.45, 133.17, 131.75, 128.52, 128.43, 128.32, 127.51, 124.79, 38.75, 36.27, 33.30, 26.58, 26.45, 23.25, 6.85. HRMS (ESI+) calc: [M+H]+ 284.2009, measured: 284.2005; difference=1.4 ppm.

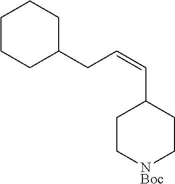 [SMILES: CC(C)(C)OC(=O)N1CCC(/C=C\CC2CCCCC2)CC1]

Tert-butyl (Z)-4-(3-cyclohexylprop-1-en-1-yl)piperidine-1-carboxylate (15): Prepared according to General Procedure D. Purified by flash chromatography (1% ethyl acetate in hexanes). Morphology: off-white oil. Purified yield: 68 mg, 74%, 91:9 Z:E (determined by quantitative 13C-NMR integration of the vinylic 13C resonances at 135.3 and 134.4 ppm, respectively). ¹H-NMR (500 MHz, CDCl₃): 5.33 (dtd, J=11.0, 7.4, 1.0 Hz, 1H), 5.20 (ddt, J=10.8, 9.2, 1.6 Hz, 1H), 4.06 (s, 2H), 2.74 (t, J=12.8 Hz, 2H), 2.38 (ddt, J=19.8, 11.6, 3.9 Hz, 1H), 1.94 (td, J=7.1, 1.6 Hz, 2H), 1.75-1.61 (m, 7H), 1.46 (s, 9H), 1.37-1.04 (m, 6H), 0.97-0.78 (m, 2H). ¹³C-NMR (126 MHz, CDCl₃) δ 154.94, 135.32, 134.38, 128.03, 127.62, 79.27, 77.16, 38.28, 35.41, 34.53, 33.29, 28.56, 26.65, 26.46. HRMS (ESI+) calc: [M+H]+ 308.2584, measured: 308.2578; difference=1.3 ppm.

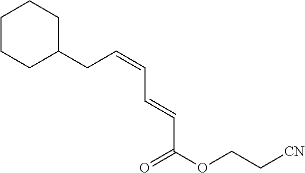 [SMILES: N#CCCOC(=O)/C=C/C=C\CC1CCCCC1]

2-Cyanoethyl (2E,4Z)-6-cyclohexylhexa-2,4-dienoate (16): Prepared according to General Procedure E. Purified by flash chromatography (10% ethyl acetate in hexanes). Morphology: clear oil. Purified yield: 51 mg, 69% yield, 86:14 Z:E. ¹H-NMR (500 MHz, CDCl₃) δ 7.64 (ddd, J=15.3, 11.6, 1.1 Hz, 1H), 6.21-6.13 (m, 1H), 5.93 (dt, J=10.9, 8.0 Hz, 1H), 5.87 (d, J=15.2 Hz, 1H), 4.35 (t, J=6.4 Hz, 2H), 2.73 (t, J=6.4 Hz, 2H), 2.19 (ddd, J=8.2, 6.8, 1.5 Hz, 2H), 1.73-1.65 (m, 3H), 1.41-1.32 (m, 1H), 1.27-1.08 (m, 4H), 1.00-0.87 (m, 2H). ¹³C-NMR (126 MHz, CDCl₃) δ 166.66, 141.80, 141.32, 127.02, 119.68, 58.61, 38.17, 36.09, 33.22, 33.17, 26.47, 26.34, 18.20. HRMS (ESI+) calc: [M+NH4]+265.1910, measured: 265.1910; difference=less than 1 ppm.

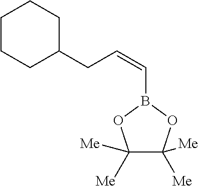 [SMILES: CC1(C)OB(/C=C\CC2CCCCC2)OC1(C)C]

(Z)-2-(3-cyclohexylprop-1-en-1-yl)-4,4,5,5-tetramethyl-1,3,2-dioxaborolane (17): Prepared according to General Procedure F. Purified by flash chromatography (5% ethyl acetate in hexanes). Morphology: clear oil. Purified yield: 63 mg, 84% yield, 94:6 Z:E. ¹H-NMR (500 MHz, CDCl₃) δ 6.44 (dt, J=14.5, 7.6 Hz, 1H), 5.36 (dt, J=13.5, 1.3 Hz, 1H), 2.30 (t, J=7.2 Hz, 2H), 1.75-1.58 (m, 5H), 1.26 (s, 16H), 1.01-0.86 (in, 2H). ¹³C-NMR (126 M Hz, CDCl₃) δ 153.93, 82.91, 77.16, 39.98, 38.31, 33.17, 26.73, 26.51, 24.98. ¹¹B-NMR (128 M Hz, CDCl₃) δ 29.82. H RMS (ASAP) calc: [M+H]+ 251.2177, measured: 251.2176; difference=0.4 ppm.

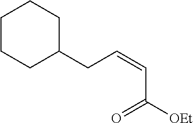 [SMILES: CCOC(=O)/C=C\CC1CCCCC1]

Ethyl (Z)-4-cyclohexylbut-2-enoate (18): Prepared according to General Procedure G. Purified by chromatography (hexanes, then linear gradient of 1 to 5% ethyl acetate in hexanes). Morphology: clear oil. Purified yield: 89 mg, 91% yield, >20:1 Z:E. ¹H-NMR (500 MHz, CDCl₃) δ 6.21 (dt, J=11.6, 7.6 Hz, 1H), 5.75 (d, J=11.6 Hz, 1H), 4.13 (q, J=7.1 Hz, 2H), 2.53 (td, J=7.2, 1.8 Hz, 2H), 1.74-1.56 (in, 5H), 1.45-1.32 (m, 1H), 1.25 (t, J=7.2 Hz, 3H), 1.22-1.07 (in, 3H), 0.95 (qd, J=12.5, 3.7 Hz, 2H). ¹³C-NMR (126 MHz, CDCl₃) δ 166.58, 149.45, 120.27, 59.75, 38.19, 36.54, 33.16, 26.49, 26.34, 14.34. HRMS (ASAP) calc: [M+H]+ 197.1536, measured: 197.1534; difference=1.0 ppm.

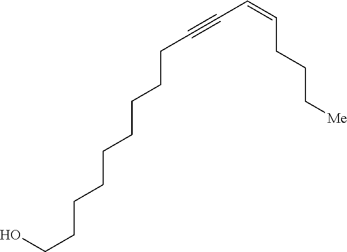 [SMILES: CCCC/C=C\C#CCCCCCCCCCO]

(Z)-heptadec-12-en-10-yn-1-ol-1-methane (20): Prepared according to General Procedure B. Purified by flash chromatography (10% ethyl acetate in hexanes). Morphology: light yellow oil. Purified yield: 45 mg, 60% yield, >20:1 Z:E. ¹H-NMR (500 MHz, CDCl₃) δ 5.80 (dt, J=10.7, 7.4 Hz, 1H), 5.42 (d, J=10.7 Hz, 1H), 3.63 (t, J=6.6 Hz, 2H), 2.32 (td, J=7.1, 2.3 Hz, 2H), 2.28 (td, J=7.3, 1.4 Hz, 2H), 1.60-1.47 (m, 4H), 1.44-1.28 (m, 12H), 0.90 (t, J=7.1 Hz, 3H). ¹³CNMR (126 MHz, CDCl₃) δ 142.72, 109.45, 94.54, 77.16, 63.19, 32.93, 31.19, 29.85, 29.63, 29.52, 29.22, 29.00, 28.98, 25.86, 22.41, 19.64, 14.05 (consistent with reported spectra: Hoye, C. et al. J. Org. Chem. 2010, 75, 7400-7403). HRMS (ASAP) calc: [M+H]+ 251.2369, measured: 251.2367; difference=0.8 ppm.

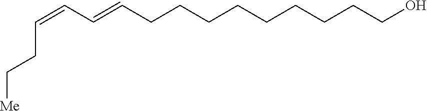 [SMILES: CCC/C=C\C=C\CCCCCCCCCO]

(10E,12Z)-hexadeca-10,12-dien-1-ol (Bombykol, 23): Prepared according to General Procedure C. Purified by flash chromatography (isocratic gradient of 15% ethyl acetate in hexanes). Morphology: clear oil. Purified yield: 59 mg, 82% yield, 84:16 Z:E. ¹H-NMR (500 MHz, CDCl₃) δ 6.36-6.22 (m, 1H), 5.97 (q, J=12.2 Hz, 1H), 5.65 (dt, J=14.6, 7.0 Hz, 1H), 5.30 (dt, J=11.1, 7.6 Hz, 1H), 3.64 (t, J=6.6 Hz, 2H), 2.10 (m, 2H), 1.64-1.20 (m, 16H), 0.92 (td, J=7.3, 1.0 Hz, 3H). ¹³C-NMR (126 MHz, CDCl₃) δ 134.81, 130.00, 128.94, 125.81, 63.24, 33.01, 32.96, 29.90, 29.69, 29.57, 29.55, 29.35, 25.88, 23.05, 13.92 (consistent with reported spectra: Kramer, M. et al. J. Agric. Food Chem. 2002, 50, 6366-6370, and Odinokov, V. et al. Chem. Nat. Compounds. 1993, 29, 668-673). HRMS (ASAP) calc: [M+H]+ 239.2369, measured: 239.2367; difference=0.8 ppm.

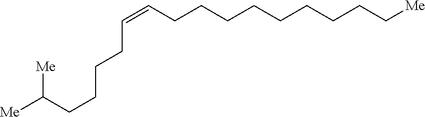 [SMILES: CCCCCCCCCC/C=C\CCCCC(C)C]

(Z)-2-methyloctadec-7-ene (25): Prepared according to General Procedure D. Purified by flash chromatography (hexanes). Morphology: clear oil. Purified yield: 88 mg, 76% yield, 90:10 Z:E (determined by quantitative 13C-NMR integration of the vinylic 13C resonances at 130.1 and 130.6 ppm, respectively). ¹H-NMR (500 MHz, CDCl₃) δ 5.35 (m, 2H), 2.06-1.98 (m, 4H), 1.52 (hept, J=6.6 Hz, 1H), 1.38-1.24 (m, 20H), 1.20-1.13 (m, 2H), 0.90-0.85 (m, 9H). ¹³C-NMR (126 MHz, CDCl₃) δ 130.08, 130.04, 77.16, 39.08, 32.08, 30.20, 29.94, 29.82, 29.81, 29.73, 29.52, 29.49, 28.14, 27.41, 27.38, 27.24, 22.85, 22.81, 14.27 (consistent with reported spectra: Chan, T. et al. J. Organomet. Chem. 1985, 285, 109-119). HRMS (ASAP) calc: [M+H]+ 267.3046, measured: 267.3041; difference=1.9 ppm.

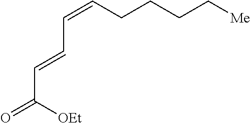 [SMILES: CCCCC/C=C\C=C\C(=O)OCC]

Ethyl (2E,4Z)-deca-2,4-dienoate (27): Prepared according to General Procedure E. Purified by flash chromatography (hexanes). Morphology: clear oil. Purified yield: 51 mg, 87% yield, 83:17 Z:E. ¹H-NMR (500 MHz, CDCl₃) δ 7.60 (ddd, J=15.2, 11.6, 1.2 Hz, 1H), 6.15-6.07 (t, J=11.6 Hz, 1H), 5.85 (d, J=15.2 Hz, 1H), 4.20 (q, J=7.1 Hz, 2H), 2.29 (q, J=8.4 Hz, 1H), 1.45-1.36 (m, 2H), 1.29 (t, J=7.1 Hz, 7H), 0.88 (td, J=6.1, 3.6 Hz, 3H). ¹³C-NMR (126 MHz, CDCl₃) δ 167.46, 141.78, 139.63, 126.56, 121.31, 60.37, 31.52, 29.19, 28.36, 22.62, 14.44, 14.12 (consistent with reported spectra: Zorin, V. et al. Chem. Nat. Compounds. 2017, 53, 1017-1019). HRMS (ESI+) calc: [M+H]+ 197.1536, measured: 197.1535; difference=0.5 ppm.

1. A method to make Z-alkene-containing compounds, the method comprising:
a. electrochemically reacting a 1-alkene with a thianthrene-containing compound to yield a first intermediate;
b. subjecting the first intermediate to an E2 elimination reaction to yield a product comprising a Z-alkenyl moiety in diastereomeric excess.

2. The method of claim 1, wherein step (a) comprises electrochemically reacting the 1-alkene in a divided cell.

3. The method of claim 1, wherein step (a) comprises electrochemically reacting the 1-alkene in an undivided cell.

4. The method of claim 1, wherein step (b) comprises treating the first intermediate with a base to drive the E2 elimination reaction.

5. The method of claim 4, wherein step (b) comprises treating the first intermediate with a weak base.

6. The method of claim 4, wherein step (b) comprises treating the first intermediate with a base selected from the group consisting of carbonates, bicarbonates, acetates, citrates, halogenated analogs of the foregoing, and combinations thereof.

7. The method of claim 1, wherein in step (b) the Z-alkenyl moiety is yielded in a diastereomeric excess of at least 50%, at least 60%, at least 70%, and least 80%, at least 90%, or at least 95%.

8. The method of claim 1, wherein in step (a), the thianthrene-containing compound is selected from the group consisting of: 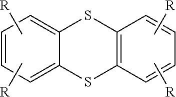
wherein each R is independently selected from the group consisting of hydrogen, halogen, hydroxy, amino, and substituted or unsubstituted, linear or branched C1-C6 alkyl.

9. The method of claim 8, wherein each R is independently selected from the group consisting of hydrogen, halogen, or methyl.

10. The method of claim 8, wherein each R is independently selected from the group consisting of hydrogen or methyl.

11. The method of claim 8, wherein each R is hydrogen.

12. The method of claim 1, wherein step (a) comprises electrochemically reacting the 1-alkene in an undivided cell in which is disposed a cathode comprising Ni foam.

13. The method of claim 1, further comprising step (c):
c. subjecting the product of step (b) to a reaction to yield a downstream product mix comprising a Z-alkenyl moiety in diastereomeric excess.

14. The method of claim 13, wherein step (c) comprises subjecting the product of step (b) to a coupling reaction.

15. The method of claim 13, wherein step (c) comprises subjecting the product of step (b) to a cross-coupling reaction.

16. The method of claim 13, wherein step (c) comprises subjecting the product of step (b) to a metal-catalyzed cross-coupling reaction.

17. The method of claim 13, wherein step (c) comprises subjecting the product of step (b) to a palladium-catalyzed cross-coupling reaction.

18. The method of claim 13, wherein in step (a), the thianthrene-containing compound is selected from the group consisting of: 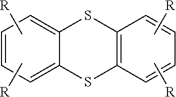
wherein each R is independently selected from the group consisting of hydrogen, halogen, hydroxy, amino, and substituted or unsubstituted, linear or branched C1-C6 alkyl.

19. The method of claim 18, wherein each R is independently selected from the group consisting of hydrogen, halogen, or methyl.

20. The method of claim 18, wherein each R is hydrogen.
